(19) United States
(12) Patent Application Publication
(10) Pub. No.: US 2025/0040890 A1
(43) Pub. Date: Feb. 6, 2025

(54) HIGH-RESOLUTION DIAGNOSTIC DATA SYSTEM FOR PATIENT RECOVERY AFTER HEART FAILURE INTERVENTION

(71) Applicant: Medtronic, Inc., Minneapolis, MN (US)

(72) Inventors: Juliana E. Pronovici, New Hope, MN (US); Shantanu Sarkar, Roseville, MN (US); Amanda D. Taylor, Roseville, MN (US); Kate Arnebeck, Shoreview, MN (US); Ekaterina B. Morgounova, Minneapolis, MN (US); Veronica Ramos, Miami, FL (US); Sean R. Landman, Minneapolis, MN (US); Hyun J. Yoon, Vadnais Heights, MN (US); Steven G. Nelson, Raynham, MA (US); Jodi L. Redemske, South Haven, MN (US)

(21) Appl. No.: 18/923,043

(22) Filed: Oct. 22, 2024

Related U.S. Application Data
(63) Continuation-in-part of application No. PCT/IB2023/053706, filed on Apr. 11, 2023.
(60) Provisional application No. 63/363,444, filed on Apr. 22, 2022.

(51) Int. Cl. A61B 5/00; A61B 5/0205; G16H 20/00

(52) U.S. Cl. CPC A61B 5/686; A61B 5/0205; G16H 20/00

(57) ABSTRACT

An example system includes an implantable medical device (IMD), processing circuitry, and communication circuitry. The IMD is configured to receive signals from sensors, determine a plurality of detected patient metrics based on the received signals, and store the plurality of determined patient metrics in the memory. The processing circuitry is to generate higher resolution diagnostic information based on the plurality of patient metrics during an initiated period of time, wherein the initiated period of time is initiated based on an intervention being provided to the patient and/or receiving a user input. The higher resolution diagnostic information is of at least one of the plurality of patient metrics and indicative of heart recovery. The communication circuitry is configured to transmit the higher resolution diagnostic information to indicate a degree of recovery for patient metrics indicative of heart recovery.

This application is a continuation-in-part of WO International Patent Application No. PCT/IB2023/053706, filed 11-4-2023, which claims the benefit of U.S. Provisional Patent Application Ser. No. 63/363,444, filed Apr. 22, 2022, the entire content of each application is incorporated herein by reference.

### FIELD

This disclosure generally relates to systems including medical devices and, more particularly, to monitoring cardiac health using such systems.

### BACKGROUND

Heart failure (HF) is a condition affecting thousands of people worldwide. Essentially, congestive HF occurs when the heart is unable to pump blood at an adequate rate in response to the filling pressure. This condition may result in congestion in the tissue, peripheral edema, pulmonary edema, and even shortness of breath. When HF is severe, it can even lead to patient death.

A variety of medical devices have been used or proposed for use to deliver a therapy to and/or monitor a physiological condition of patients. Some medical devices have been used or proposed for use to monitor HF or to detect HF events. HF interventions may include electrical stimulation therapy and drug therapy. For example, patients suffering from or at risk for HF may be treated with diuretic agents and/or angiotensin converting enzyme inhibitors. In addition, patients may be treated with nitroglycerin to reduce the symptoms of HF. Even though interventions are available, patients with other cardiac conditions may be at greater risk of severe complications with the conditions of HF.

### SUMMARY

Generally, this disclosure describes techniques performed by a computer system for processing diagnostic information that is indicative of a degree of heart recovery to improve patient outcomes. An implantable medical device (IMD), such as an insertable cardiac monitor (ICM), may automatically generate and store patient data. The patient data may include therapy use statistics (e.g., pacing or shocks), impedance, subcutaneous impedance, intracardiac impedance, thoracic impedance, heart rate, heart rate variability, patient activity, respiratory rate, r-wave amplitude, heart sounds, tissue oxygenation, and other patient metrics. A patient who has HF decompensation may undergo HF interventions, such as hospitalization or remote care, medication change (e.g. change in amount of diuretic), and diet change (e.g. change in salt intake) to help the heart recover. During a period of time after the intervention is administered to the patient, higher resolution diagnostic information based on one or more patient metrics may be transmitted to determine a degree the heart recovers after receiving the intervention. This higher resolution diagnostic information may include raw data collected to determine values for the patient metrics. The higher resolution diagnostic information may be aggregated hourly, transmitted at least several times a day, and may be used to determine whether the intervention is effective, needs to be altered to improve effectiveness or safety of the intervention, and/or needs to be stopped because the heart has recovered above a threshold or the intervention is causing harm to the patient.

Generating higher resolution diagnostic information and transmitting the generated higher resolution diagnostic information to indicate a degree of recovery for one or more patient metrics indicative of heart recovery helps provide an accurate indication of a degree of recovery in a shorter period of time, for example four days, after intervention. For example, the generation of higher resolution diagnostic information to indicate a degree of recovery helps provide an accurate degree of recovery shortly after intervention so patient treatments may be adjusted, if necessary, at a much earlier time after intervention, which improves patient care and helps with the treatment of patients experiencing negative health events such as HF.

The techniques of this disclosure may be implemented by systems including one or more IMDs and computing devices that can autonomously and continuously collect physiological parameter data while the IMD is implanted in a patient over months or years and perform numerous operations per second on the data to enable the systems herein to determine the health status of a patient. Using techniques of this disclosure with an IMD may be advantageous when a physician cannot be continuously present with the patient to evaluate the physiological parameters and/or where performing the operations on the data described herein could not practically be performed in the mind of a physician.

In some examples, the techniques and systems of this disclosure may use a machine learning model to more accurately infer the patient's condition, e.g., to determine a degree of recovery from a treatment, based on physiological data collected by an IMD. In some examples, the machine learning model is trained with a set of training instances, where one or more of the training instances comprise data that indicate relationships between various sets of input data and outputs. Because the machine learning model is trained with potentially thousands or millions of training instances, the machine learning model may reduce the amount of error in risk level or other values useful for control of dialysis. Reducing errors using the techniques of this disclosure may provide one or more technical and clinical advantages, such as increasing the efficacy of therapies prescribed based on the output of the machine learning model.

In one example, this disclosure describes a method of obtaining a plurality of detected patient metrics within an implantable medical device of a patient; generating higher resolution diagnostic information of the patient during an initiated period of time, wherein the initiated period of time is initiated based on one or more of an intervention being provided to the patient and receiving a user input, such as from a clinician or a patient, and the higher resolution diagnostic information is of at least one of the plurality of patient metrics and indicative of heart recovery; and transmitting the generated higher resolution diagnostic information to indicate a degree of recovery for one or more patient metrics indicative of heart recovery.

In another example, this disclosure describes a system comprising an implantable medical device comprising one or more sensors, and configured to receive one or more signals from the sensors, to determine a plurality of detected patient metrics based at least in part on the received signals; processing circuitry to generate higher resolution diagnostic information based on at least one of the plurality of patient metrics during an initiated period of time, wherein the initiated period of time is initiated based on one or more of an intervention being provided to the patient and receiving a user input, such as from a clinician or a patient, and the higher resolution diagnostic information is of at least one of the plurality of patient metrics and indicative of heart recovery; and communication circuitry configured to transmit the higher resolution diagnostic information to indicate a degree of recovery for one or more patient metrics indicative of heart recovery.

In another example, this disclosure describes a non-transitory computer-readable storage medium comprising instructions that, when executed by processing circuitry, cause the processing circuitry to store a plurality of detected patient metrics within an implantable medical device of a patient; generate higher resolution diagnostic information of the patient during an initiated period of time, wherein the initiated period of time is initiated based on one or more of an intervention being provided to the patient and receiving a user input, such as from a clinician or a patient, and the higher resolution diagnostic information is of at least one of the plurality of patient metrics and indicative of heart recovery; and transmit the generated higher resolution diagnostic information to a display to indicate a degree of recovery for a respective patient metric indicative of heart recovery.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured to transmit higher resolution diagnostic information, generate a degree of patient metric recovery and/or generate a degree of heart recovery in accordance with one or more techniques of this disclosure.

FIG. 2 is a block diagram illustrating an example configuration of a patient sensing device that operates in accordance with one or more techniques of the present disclosure.

FIG. 3 is a block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a block diagram illustrating an example system that includes an implantable medical device used to obtain diagnostic information of various patient metrics.

FIG. 6 is a flow diagram illustrating an example operation to transmit higher resolution diagnostic information, generate a degree of patient metric recovery and/or generate a degree of heart recovery.

FIG. 7 is a flow diagram illustrating an example operation to determine whether to generate a degree of HF risk or generate a degree of patient metric recovery and/or a degree of heart recovery.

FIG. 8 is a conceptual diagram illustrating an example machine learning model configured to determine one or more of dialysis parameters, target dry weight, fluid volume, or risk of a dialysis session based on impedance measurements.

FIG. 9 is a conceptual diagram illustrating an example training process for an artificial intelligence model, in accordance with examples of the current disclosure.

FIG. 10A is a perspective drawing illustrating an example IMD.

FIG. 10B is a perspective drawing illustrating another example IMD.

FIG. 11A is a graph showing patient daily activity of a patient versus days from an intervention provided to the patient.

FIG. 11B is a graph showing daily night heart rate of a patient versus days from an intervention provided to the patient.

FIG. 11C is a graph showing daily heart rate variability of a patient versus days from an intervention provided to the patient.

FIG. 11D is a graph showing various patient data of a patient versus days from an intervention provided to the patient.

Like reference characters refer to like elements throughout the figures and description.

### DETAILED DESCRIPTION

A variety of types of implantable and external devices are configured to detect diagnostic information, and, in some cases, other physiological signals, indicative of HF. External devices that may be used to non-invasively sense and monitor diagnostic information and other physiological signals include wearable devices with electrodes configured to contact the skin of the patient, such as patches, watches, rings, necklaces, hearing aids, clothing, car seats, or bed linens. External devices may include smartphones, desktop, laptop, or tablet computers, or workstations associated with such systems or entities, or employees of such systems or entities. Such external devices may facilitate monitoring of patient health during normal daily activities.

Congestive HF may occur gradually over time due to heart disease, patient inactivity, cardiac arrhythmias, hypertension, and other conditions. A relatively rapid worsening of the patient's condition, e.g., a decompensation, may precipitate hospitalization and, in some cases, death.

A patient who has HF may be provided intervention, such as hospitalization, medication change (e.g., change in amount of diuretic), cardiac rehabilitation, exercise, increased activity, and diet change (e.g., change in salt intake) to hopefully help the heart recover. Some other examples of HF interventions may include electrical stimulation therapy and various drug therapies. For example, patients suffering from or at risk for HF may be treated with diuretic agents and/or angiotensin converting enzyme inhibitors. In addition, patients may be treated with nitroglycerin to reduce the symptoms of HF. Even though interventions are available, patients with other cardiac conditions may be at greater risk of severe complications with the conditions of HF. Accordingly, hearts of various patients may recover at different rates and at different amounts depending on many variables.

Implantable medical devices (IMDs) may sense and monitor patient metrics and detect status of health conditions of the patient that may indicates conditions such as HF and recovery from HF. In some examples, diagnostic information may be stored in an IMD. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ or LINQ II™ Insertable Cardiac Monitor (ICM), available from Medtronic, Inc., of Minneapolis, Minnesota, which may be inserted subcutaneously. Another example of such an IMD is the Micra™ leadless pacemaker, available from Medtronic, Inc. Such IMDs may facilitate relatively longer-term monitoring of patients during normal daily activities, and may periodically transmit collected data to a remote patient monitoring system, such as the Medtronic Carelink™ Network.

An IMD, e.g., a pacemaker, cardioverter and/or defibrillator, or a monitor that does not provide intervention, may generate and store patient data regarding patient metrics. The patient metrics may include, as examples, fluid level, heart rate, respiration rate, patient activity, temperature, heart sounds, oxygenation, and R-wave morphological characteristics. Other example patient metrics include coughing, speech, posture, tissue perfusion, hematocrit, thoracic impedance, subcutaneous impedance, intracardiac impedance, heart rate variability, weight, blood pressure, sleep apnea burden (which may be derived from respiration rate), ischemia burden, sleep duration, sleep quality, PVC burden, the occurrence, frequency or duration cardiac events, and sensed cardiac intervals (e.g., heart rate or Q-T intervals). Examples of cardiac events may include atrial and ventricular tachyarrhythmias. Another example patient metric is the ventricular rate during atrial fibrillation. The concentration or levels of various substances, such as blood glucose, hematocrit, troponin and/or brain natriuretic peptide (BNP) levels, within the patient may also be used as one or more patient metrics.

The IMD may provide diagnostic information to one or more users via one or more devices, such as IMD programmers or other computing devices. The diagnostic information may be related to, generated from, or may even include the one or more patient metrics. The diagnostic information may include, as examples, values of the patient metrics and raw data used to derive the values of the patient metrics.

The IMD may provide different resolutions of such diagnostic information, such as providing high resolution diagnostic information or low resolution diagnostic information based on intervention being provided to a patient or receiving a user input. In some examples, an IMD may provide lower resolution diagnostic information over a longer period of time for various reasons, such as saving power and limiting usage of memory. In some examples, an IMD may provide lower resolution diagnostic information when a patient has not experienced a recent change in diagnosis or treatment. In some examples, lower resolution diagnostic information may be detected and collected monthly, weekly, or daily.

Higher resolution diagnostic information based on one or more patient metrics may be transmitted to a user to aid the evaluation of HF intervention and to determine whether intervention is working, may need to be altered to improve heart recovery, and/or may need to stop due to the degree of heart recovery being great enough. The resolution of the data may refer to how often the data is determined and stored, with higher resolution data being stored more frequently. The resolution of data may also refer to whether the data includes raw values of measured patient metrics, such as an impedance, or values derived from such raw values, with raw values being of higher resolution than derived values. In one example, higher resolution diagnostic information may be in the form of raw data, e.g., an impedance, which is transmitted at least several times a day. Higher resolution diagnostic information may include frequently collected patient metrics such that the resolution may allow for relatively continuous monitoring of the patient. In some examples, higher resolution diagnostic information may be detected and collected hourly. In some examples, higher resolution diagnostic information may be detected and collected every 5 minutes. Lower resolution diagnostic data may be detected and collected at a lower rate than higher resolution diagnostic information. In some examples, lower resolution diagnostic information may be detected and collected daily, weekly, monthly, etc.

The IMD may provide higher resolution diagnostic information not readily available from sources other than the IMD, e.g., such as external bed-side monitors, laboratory tests, or various imaging modalities.

When a heart begins to recover from HF after intervention, some diagnostic parameters may improve to indicate the patient is improving. Some examples of changes to physiological parameters of a patient to indicate improvement may include increasing impedance, decreasing resting heart rate, decreasing respiratory rate, increasing activity, increasing r-wave amplitude, reducing amplitude of S3 heart sounds or increasing amplitude and slew of S1 heart sounds, improving tissue oxygenation, increasing heart rate variability, decreasing night heart rate, changes in accelerometer signals, changes in optical signals. However, clinicians may have few objective data to accurately evaluate heart recovery in the short term or acutely after providing an intervention.

In some examples, a degree of heart recovery may be generated based on the generated higher resolution diagnostic information. In some examples, the degree of heart recovery may be automatically generated. The higher resolution diagnostic information may be aggregated over a variety of time periods such as hourly, every 5 minutes, etc. and may be transmitted at least several times a day over a length of time, for example 7-14 days, and may be used to indicate a degree of heart recovery to determine whether the intervention is effective, needs to be altered to improve effectiveness, and/or needs to be stopped because the heart has recovered above a threshold.

This disclosure describes techniques for generating and transmitting diagnostic information that is indicative of a degree of heart recovery for a period of time. More particularly, that is indicative of a period of time after an HF intervention is administered and/or a user input is received. More particularly, the disclosure describes techniques for storing a plurality of automatically detected patient metrics within an IMD, generating higher resolution diagnostic information of the patient during an initiated period of time, and transmitting the generated higher resolution diagnostic information to indicate a degree of recovery for one or more patient metrics indicative of heart recovery. In some examples, a degree of patient metric recovery may indicate heart worsening, such as a degree of patient metric recovery being negative when a value of a patient metric worsens over a period of time. In some examples, a degree of heart recovery may indicate heart worsening, such as a degree of heart recovery being negative when a value of a heart recovery worsens over a period of time.

Certain conditions may be automatically monitored and used to create a degree of heart recovery that a clinician may review periodically, or that may be automatically transmitted to a clinician when the degree of heart recovery indicates that the patient is improving at a desired rate, not improving at the desired rate, not improving, or getting worse. Using the degree of recovery for one or more patient metrics and/or the degree of heart recovery, a clinician or other healthcare professional may alter or titrate the intervention of the patient to provide the patient with a better course of intervention that may improve heart recovery.

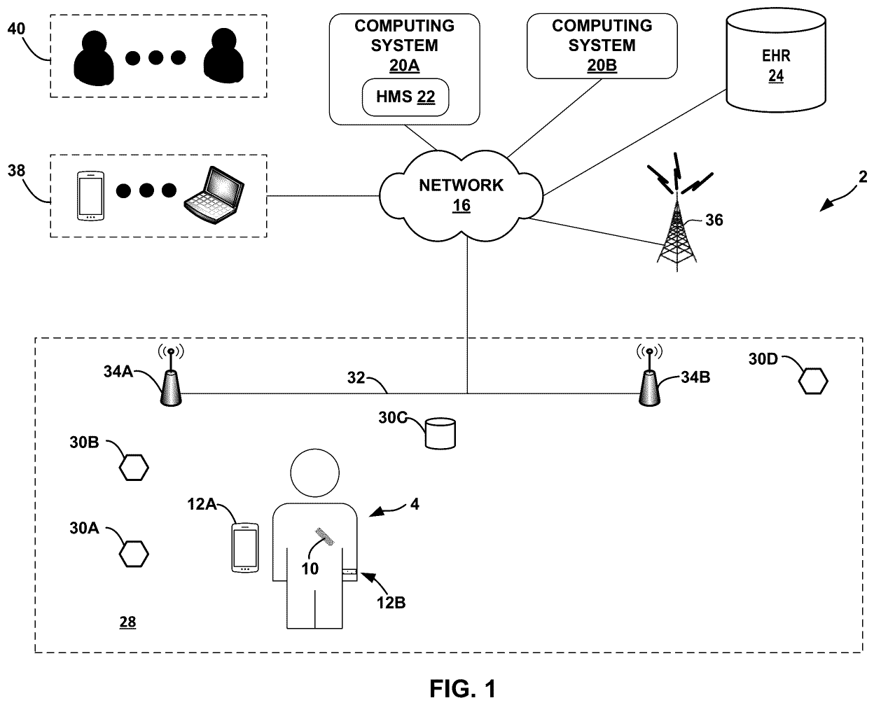

FIG. 1 is a block diagram illustrating an example system 2 configured indicate a degree of recovery for one or more patient metrics of patient 4 and/or indicate a degree of heart recovery of patient 4. The example techniques may be used with one or more patient sensing devices, e.g., IMD 10, which may be in wireless communication with one or more patient computing devices, e.g., patient computing devices 12A and 12B (collectively, “patient computing devices 12”). Although not illustrated in FIG. 1, IMD 10 include electrodes and other sensors to sense physiological signals of patient 4 to detect patient metrics, and may collect and store detected patient metrics, generate higher resolution diagnostic information of one or more of the detected patient metrics, and transmit the higher resolution data to indicate a degree of recovery for one or more patient metrics, and/or generate a degree of heart recovery based on the data. In some examples, the degree of heart recovery may be automatically generated.

After intervention is provided to patient 4, there may be particular patient metrics that may provide a greater indication of whether heart is recovering after intervention. In some examples, patient metrics may include one or more of fluid level, heart rate, respiration rate, patient activity, heart rate variability, night heart rate, temperature, heart sounds, oxygenation, and R-wave morphological characteristics. In some examples, when an intervention is provided because of a HF diagnosis if patient 4 is recovering after the intervention is provided, patient 4 may have one or more of increasing impedance, increasing patient activity, increasing heart rate variability, decreasing night heart rate, decreasing heart rate, decreasing respiratory rate, increasing activity, and increasing R-wave amplitude. One or more of these metrics may indicate to a clinician that patient 4 is improving after intervention.

One or more of how much the metrics have improved, the rates the metrics have improved, how many metrics have improved, and how much the metrics have improved comparted to a threshold of expected improvement may indicate a degree of heart recovery for patient 4.

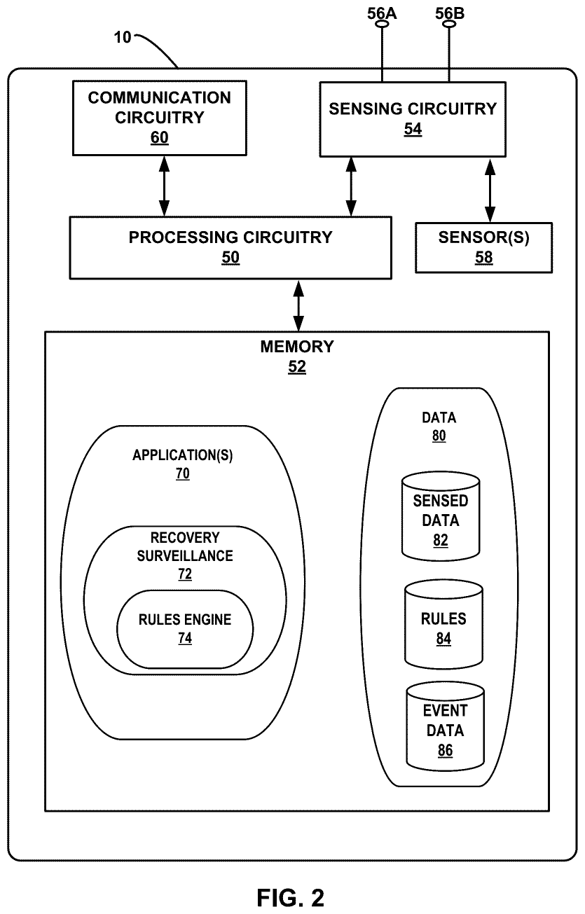

As shown in FIGS. 2, 3, and 5, processing circuitry 50, 130, 23 is located in a respective IMD 10, patient computing devices 12, and computing systems 20. For simplicity, processing circuitry 50 will be referenced in the examples discussed below, but any one or more of processing circuitries 50, 130, and 23 may be used as the processing circuitry. As shown in FIGS. 2, 3, and 5, communication circuitry 60, 140, 24 is located in a respective IMD 10, patient computing devices 12, and computing systems 20. For simplicity, communication circuitry 60 will be referenced in the examples discussed below, but any one or more of communication circuitries 60, 140, and 24 may be used as the processing circuitry.

In some examples, in system 2 processing circuitry and communication circuitry may be located in IMD 10, such as processing circuitry 50 and communication circuitry 60. In some examples, one or more of processing circuitry and communication circuitry may be located external to IMD 10, such as processing circuitry 130, 23 and communication circuitry 140, 24.

In some examples, the detected patient metrics may be stored in one or more of IMD 10, computing device(s) 12, and/or computing system 20, such as memory 52. In some examples, communication circuitry 60 may transmit generated high resolution diagnostic information to clinician computing device 38 to indicate a degree of heart recovery to a clinician. The transmitted higher resolution diagnostic information may be displayed on clinician computing device 38. In some examples, communication circuitry 60 may transmit a degree of recovery for one or more patient metrics and/or transmit a degree of heart recovery to clinician computing device 38 to be displayed on clinician computing device 38 to indicate an amount the heart of patient 4 has recovered over a period of time after intervention. Accordingly, a clinician 40 may be able to determine, based on received higher resolution diagnostic information and/or received degree of heart recovery, whether the intervention is effective, needs to be altered to improve effectiveness, and/or needs to be stopped because the heart has recovered above a desired threshold. The higher resolution diagnostic information may be aggregated over a variety of time periods such as hourly, every 5 minutes, etc. and may be transmitted at least several times a day over a length of time, for example 7-14 days.

During a period of time after intervention is administered to patient 4, processing circuitry 50 may generate higher resolution diagnostic information of one or more patient metrics to indicate a degree of heart recovery after patient 4 is provided the intervention. This higher resolution diagnostic information may include raw data collected by IMD 10 to determine values for the patient metrics. In some examples, the period of time to generate higher resolution diagnostic information may be initiated by when intervention is provided to patient. In some examples, higher resolution diagnostic information may be generated continuously in the background and be shown upon request, such as by a clinician. In some examples, the period of time to generate higher resolution diagnostic information may initiated when an ambulatory HF prediction risk score is above a threshold and shows a patient is at high risk. In some examples, a time an intervention is provided to patient 4 may include when intervention is first provided to patient 4, when the intervention to patient 4 is completed, or at any point between when the intervention is first provided to patient 4 to when the intervention to patient 4 is completed.

In some examples, processing circuitry 50 may generate lower resolution diagnostic information of one or more patient metrics before intervention is administered to patient 4, such as when monitoring patient 4 for HF risk. Lower resolution diagnostic information may be detected and collected at a lower rate than higher resolution diagnostic information. In some examples, lower resolution diagnostic information may be detected and collected daily, weekly, monthly, etc. In some examples, in response to intervention being administered to patient 4, processing circuitry 50 may switch from generating lower resolution diagnostic information of one or more patient metrics to generating higher resolution diagnostic information of one or more patient metrics. In some examples, processing circuitry 50 may generate higher resolution diagnostic information for a period of time such as 3-days, 7-days, 14-days, and 21-days. The length of the period of time may be any other period of time that is of interest, such as of interest to clinician 40 and/or of interest to patient 4. In some examples, in response to the period of time ending (e.g. 14 days), processing circuitry 50 may switch back to generating lower resolution diagnostic information of one or more patient metrics and/or stop generating higher resolution diagnostic information. In some examples, a length of the period of time may be based on a type of detected patient metric being compared by processing circuitry 50. In some examples, processing circuitry 50 may switch back to generating lower resolution diagnostic information of one or more patient metrics and/or stop generating higher resolution diagnostic information in response to a user input, such as a clinician. In some examples, processing circuitry 50 may switch back to generating lower resolution diagnostic information of one or more patient metrics and/or stop generating higher resolution diagnostic information in response to determining a generated degree of heart recovery and/or patient metric recovery is above a threshold.

In some examples, the period of time to generate higher resolution diagnostic information may be initiated by when user inputs a time to initiate generating higher resolution diagnostic information. In some examples, user may be clinician 40 or patient 4. In some examples, this could be a period of time after intervention has begun and/or after intervention is completed to monitor heart recover over a period of time of interest. Some examples of the length of the period of time may be 3-days, 7-days, 14-days, and 21-days. The length of the period of time may be any other period of time that is of interest, such as of interest to clinician 40 and/or of interest to patient 4.

In some examples, processing circuitry 50 may compare the plurality of detected patient metrics detected at an end of the initiated period of time to a respective detected patient metric detected at a beginning of the initiated period of time and then generate a degree of heart recovery based on the comparison. In some examples, the degree of heart recovery may be automatically generated. The comparison compares the automatically detected patient metrics at a particular time after the beginning of the initiated period (e.g. end of the initiated period of time), such as 3-days, 7-days, or 14-days after the initiated period of time, to the detected patient metrics at the beginning of the initiated period of time. The difference between the detected patient metrics at the two different times may indicate a degree of heart recovery over the period of time.

In some examples, the beginning of the initiated period may be when HF intervention begins, such as changing an amount of diuretic given to a patient, and the end of the initiated period of time may be an amount of time, such as 3-days, 7-days, 10-days, or any other amount of time of interest after the beginning of the initiated period. In some examples, the end of the initiated time may be selected by a user, such as a clinician 40. In some examples, the end of the initiated time may be determined by one or more of the type of intervention be provided, a condition of the heart at the intervention is initiated, age of patient, and/or other physiological parameters of patient 4, such as patient metrics detected by IMD 10.

In some examples, processing circuitry 50 may generate an average of each of the plurality of automatically detected patient metrics detected over the initiated period of time. One or more of IMD 10, computing device(s) 12, and/or computing system 20 may compare each of the averages to the patient metrics detected at a beginning of the initiated period of time, and then generate a degree of recovery based on the comparison. In some examples, the degree of heart recovery may be automatically generated. In some examples, the comparison may compare the averages of the detected patient metrics over a period of time, such as 3-days, 7-days, or 14-days after the initiated period of time, to the detected patient metrics at the beginning of the initiated period of time. The difference between the average of detected patient metrics over a period of time and the detected patient metrics at the beginning of the period of time may indicate a degree of heart recovery over the period of time.

In some examples, processing circuitry 50 may generate an average of each of the plurality of detected patient metrics detected over a second period of time, the second period of time beginning after the initiated period of time is initiated. Processing circuitry 50 may compare each of the averages to the detected patient metric detected at a beginning of the initiated period of time, and then generate a degree of recovery based on the comparison. In some examples, the degree of heart recovery may be automatically generated. In some examples, the initiated period of time and the second period of time end at the same time. In some examples, the comparison may compare the averages of the detected patient metrics over the second period of time, such as 3-days, to the detected patient metrics at the beginning of the initiated period of time, which may be 7-days before the end of the time periods (e.g. 4-days before the beginning of the second period of time in this example). The difference between the average of detected patient metrics over the second period of time and the detected patient metrics at the beginning of the initiated period of time may indicate a degree of heart recovery over the period of time. In some examples, a proportion of change relative to the patient metrics at the beginning of the time period may indicate a degree of heart recovery over the period of time. In some examples, a relative difference between the detected patient metrics and a baseline measurement of the patient, for example 60 days prior, may indicate a degree of heart recovery over the period of time. In some examples, comparing a temporal recovery trajectory to an expected recovery trajectory indicate a degree of heart recovery over the period of time.

In some examples, the initiated period of time and the second period of time end at the same time. In some examples, the comparison may compare the averages of the detected patient metrics over the second period of time, such as 3-days, to the detected patient metrics at the beginning of the initiated period of time, which may be 7-days before the end of the time periods (e.g., 4-days before the beginning of the second period of time in this example). The difference between the average of detected patient metrics over the second period of time and the detected patient metrics at the beginning of the initiated period of time may indicate a degree of heart recovery over the period of time.

In some examples, processing circuitry 50 may generate a rate of change of each of the plurality of detected patient metrics detected over the initiated period of time, and compare the rate of each of the rates of change to a respective threshold level. In some examples, processing circuitry 50 may generate a degree of patient metric recovery of one or more of the plurality of detected patient metrics based on the comparison. In some examples, processing circuitry 50 may generate a degree of patient metric recovery of each of the plurality of detected patient metrics based on the comparison. In some examples, the degree of patient metric recovery may be automatically generated. The degree of patient metric recovery may indicate an amount each parameter recovered over the initiated period of time compared to an expected amount of recovery. In some examples, processing circuitry 50 may generate a degree of heart recovery based on the generated degrees of patient metric recovery, the degree of heart recovery may indicate an amount the heart recovered over the initiated period of time.

In some examples, processing circuitry 50 may generate a proportion of change of detected patient metrics relative to the detected patient metrics at the beginning of the time period to indicate a degree of heart recovery over the period of time. In some examples, processing circuitry 50 may generate a relative difference between the detected patient metrics and a baseline measurement of the patient metrics, for example taken 60 days prior, to indicate a degree of heart recovery over the period of time. In some examples, processing circuitry 50 may generate a comparison of a temporal recovery trajectory to an expected recovery trajectory to indicate a degree of heart recovery over the period of time. The expected recovery trajectory may be based on a type of intervention that is provided to the patient.

In some examples, processing circuitry 50 may generate a degree of heart recovery based on one or more of the comparisons discussed in the examples above.

In some examples, a degree of heart recovery may be determined based on a predetermined number of patient metrics exceeding their representative thresholds or a weighted score for each of the patient metrics for exceeding one or more thresholds. Additionally or alternatively, a degree of heart recovery may be determined by prediction/probability modeling using the values or stratified states of each detected patient metric. In some examples, the prediction and/or probability modeling according to the techniques described herein may include Bayesian Belief Networks (BBN) or Bayesian machine learning (ML) models (these sometimes referred to as Bayesian Networks or Bayesian frameworks herein), Markov random fields, graphical models, artificial intelligence (AI) models (e.g., Naive Bayes classifiers or deep learning models), and/or other belief networks, such as sigmoid belief networks, deep belief networks (DBNs), etc. In other examples, the disclosed technology may leverage non-Bayesian prediction or probability modeling, such as frequentist inference modeling or other statistical models. In addition, known model-selection techniques, such as Bayesian information criterion (BIC) or Akaike information criterion (AIC), may be used to evaluate probability models prior to use.

In some examples, a degree of heart recovery may be determined by the sum, average, or other combination of weighted scores for each of the patient metrics. Each patient metric may have one or more metric-specific thresholds that stratify the state of the metric. Since some states or metrics may be more indicative of heart recovery, these states and/or metrics may provide a greater contribution to the determined degree of heart recovery. For example, an increase in impedance may have a weighted score that is double that of an increase for patient activity. In other words, impedance may be a greater degree of heart recovery factor to the patient than patient activity. Alternatively, a probability analysis may be performed on some or all of the patient metrics to determine the probability that the heart of patient 4 will continue to recover. For example, a probability model may be applied to the values of the detected patient metrics to determine the level, e.g., the probability, that patient 4 will recover from HF. In some examples, a patient's history of recovery may be adaptively incorporated to adjust threshold for recovery over time. In some examples, a rate of prior HF hospitalizations may be adaptively incorporated to adjust threshold for recovery over time.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 takes the form of the LINQ II™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. In some examples, IMD 10 takes the form of the Micra™. Furthermore, although described primarily in the context of examples including a single implanted patient sensing device, in some examples a system includes one or more patient sensing devices, which may be implanted within patient 4 or external to (e.g., worn by) patient 4. For example, a system with two IMDs 10 may capture different values of a common patient parameter with different resolution/accuracy based on their respective locations. In some examples, instead of or in addition to two IMDs 10, system 2 may include a ventricular assist device or WAED in addition to IMD 10.

Patient computing device(s) 12 are configured for wireless communication with IMD 10. Computing device(s) 12 retrieve sensed data and other sensed physiological data from IMD 10 that was collected and stored by the IMD. In some examples, computing device(s) 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing device(s) 12 may communicate with IMD 10 and each other according to the Bluetooth® or Bluetooth® Low Energy (BLE) protocols, as examples. In some examples, only one of computing device(s) 12, e.g., computing device 12A, is configured for communication with IMD 10, e.g., due to execution of software (e.g., part of a health monitoring application as described herein) enabling communication and interaction with an IMD.

In some examples, computing device(s) 12, e.g., wearable computing device 12B in the example illustrated by FIG. 1, may include electrodes and other sensors to sense physiological signals of patient 4, and may collect and store physiological data and detect episodes based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A.

One or more of computing device(s) 12 may be configured to communicate with a variety of other devices or systems via a network 16. For example, one or more of computing device(s) 12 may be configured to communicate with one or more computing systems, e.g., computing systems 20A and 20B (collectively, “computing systems 20”) via network 16. Computing systems 20A and 20B may be respectively managed by manufacturers of IMD 10 and computing device(s) 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 may help generate a degree of heart recovery of patient 4 by system 2.

Computing device(s) 12 may transmit data, including data retrieved from IMD 10, to computing systems 20 via network 16. The data may include sensed data, e.g., values of physiological parameters measured by IMD 10 and, in some cases one or more of computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing device(s) 12 to generate a degree of heart recovery for patient 4. In some examples, HMS 22 provides data from EHR 24 to computing device(s) 12 and/or IMD 10 for storage therein and use as part of their algorithms for generating a degree of heart recovery.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to generate diagnostic information of patient 4, such as high resolution diagnostic information, based on data sensed by IMD 10. In some examples, IMD 10 may be configured to generate a degree of heart recovery based on data sensed by IMD 10, and, in some cases, other data, such as data sensed by computing devices 12A and/or 12B, and data from EHR 24.

In some examples, IMD 10 may be configured to transmit data, such as sensed, measured, and/or determined values of diagnostic parameters (e.g., heart rates, impedance measurements, impedance scores, fluid indices, respiratory rate, activity data, cardiac electrograms (EGMs), historical physiological data, blood pressure values, etc.), to wireless access points 34 and/or computing device(s) 12. Wireless access points 34 and/or computing device(s) 12 may then communicate the retrieved data to computing systems 20 via network 16.

In some examples, IMD 10 may be configured to transmit data over a wired or wireless connection to computing system 20 or to computing device(s) 12. For example, computing device(s) 12 may receive data from IMD 10 or from computing device(s) 12. In another example, computing device(s) 12 may receive data from computing system 20 or from medical IMD 10, such as physiological parameter values, diagnostic states, or probability scores, via network 16. In such examples, computing device(s) 12 may determine the data received from computing system 20 or from IMD 10 and may store the data to a storage device in the computing device(s) accordingly.

In addition, IMD 10 may serve as or include data server(s). In other examples, computing system 20 may communicate with each of IMD 10 via a wired or wireless connection, to receive physiologic parameter values or diagnostic states from IMD 10. In some examples, physiological parameter values may be transferred from IMD 10 to computing system 20 and/or to computing device(s) 12.

In some cases, computing system 20 may be configured to provide a secure storage site for data that has been collected from IMD 10 and/or computing device(s) 12. In some instances, computing system 20 may include a database that stores medical- and health-related data. For example, computing system 20 may include a cloud server or other remote server that stores data collected from IMDs 10 and/or computing device(s) 12. In some cases, computing system 20 may assemble data in web pages or other documents for viewing by trained professionals, such as clinicians, via clinician computing devices 38. One or more aspects of the example system described with reference to FIG. 1 may be implemented with general network technology and functionality, which may be similar to that provided by the Medtronic CareLink® Network.

In some examples, one or more of clinician computing devices 38 may be a tablet or other smart device located with a clinician, by which the clinician may program, receive alerts from, and/or interrogate IMD 10. For example, the clinician may access data collected by IMD 10 through a clinician computing device 38, such as when patient 4 is in between clinician visits, to check on a status of a medical condition. In some examples, the clinician may enter instructions for a medical intervention for patient 4 into an application executed by clinician computing device 38, such as based on a degree of patient metric recovery and/or a degree of heart recovery determined by IMD 10, computing device(s) 12, computing system 20, or any combination thereof, or based on other patient data known to the clinician.

One clinician computing device 38 may transmit instructions for medical intervention to another of clinician computing devices 38 located with patient 4 or a caregiver of patient 4. For example, such instructions for medical intervention may include an instruction to change a drug dosage, timing, or selection, to schedule a visit with the clinician, or to seek medical attention. In some examples, instructions for medical intervention may be adjusted by the clinician based on a preference of the clinician. In some examples, clinician computing devices 38 may receive questions for a clinician to ask patient 4 based on detected patient metrics, degree of patient metric recovery and/or degree of heart recovery. In some examples, the questions may come from a manufacturer, a clinician community, and/or are crowdsourced. In further examples, a clinician computing device 38 may generate an alert to patient 4 (or relay an alert determined by IMD 10, computing device(s) 12, or computing system 20) based on a probability score (e.g., posterior probability) determined from physiological parameter values of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

To generate a degree of heart recovery, IMD 10 may apply rules to the data, which may be referred to as patient parameter data. In response to generating higher resolution diagnostic data during a time period, IMD 10 may wirelessly transmit a message to one or both of computing devices 12A and 12B. The message may indicate that IMD 10 generated higher resolution diagnostic data during an initiated time period. The message may indicate a time that IMD 10 generated higher resolution information. The message may include the generated higher resolution diagnostic information based on at least one of a plurality of patient metrics. The message may include a generated degree of heart recovery.

As will be described herein, processing circuitry of one or more devices of system 2 may be configured to generate a degree of heart recovery based at least in part on higher resolution diagnostic information of patient 4 based on data sensed by IMD 10 and, in some cases, other data, such as data sensed by computing devices 12A and/or 12B, and data from EHR 24. To generate a degree of heart recovery, the processing circuitry may apply rules to the data, which may be referred to as patient metrics. In response to generation of a degree of heart recovery, communication circuitry may wirelessly transmit a message to one or both of computing devices 12A and 12B. The message may indicate that processing circuitry 50 generated a degree of heart recovery of patient 4. The message may indicate a time that processing circuitry 50 generated a degree of heart recovery. The message may include patient metrics collected by IMD 10, e.g., data which lead to generation of a degree of heart recovery. The patient metrics may include values of one or more patient metrics and/or digitized patient metrics.

For example, environment 28 may include one or more Internet of Things (IoT) devices, such as IoT devices 30A-30D (collectively “IoT devices 30”) illustrated in the example of FIG. 1. IoT devices 30 may include, as examples, so called “smart” speakers, cameras, televisions, lights, locks, thermostats, appliances, actuators, controllers, or any other smart home (or building) devices. In the example of FIG. 1, IoT device 30C is a smart speaker and/or controller, which may include a display. IoT devices 30 may provide audible and/or visual alarms when configured with output devices to do so. In some examples, IoT devices 30 that include cameras or other sensors may activate those sensors to collect data regarding patient 4, e.g., for evaluation of the condition of patient 4.

Computing device(s) 12 may be configured to wirelessly communicate with IoT devices 30 to cause IoT devices 30 to take the actions described herein. In some examples, HMS 22 communicates with IoT devices 30 via network 16 to cause IoT devices 30 to take the actions described herein, e.g., in response to receiving the message from computing device(s) 12 as described above. In some examples, IMD 10 is configured to communicate wirelessly with one or more of IoT devices 30, e.g., in response to detection of a degree of heart recovery when communication with computing device(s) 12 is unavailable. In such examples, IoT device(s) 30 may be configured to provide some or all of the functionality ascribed to computing device(s) 12 herein.

Environment 28 includes computing facilities, e.g., a local network 32, by which computing device(s) 12, IoT devices 30, and other devices within environment 28 may communicate via network 16, e.g., with HMS 22. For example, environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access points 34A and 34B (collectively, “wireless access points 34”) that provide support for wireless communications throughout environment 28. Additionally or alternatively, e.g., when local network is unavailable, computing device(s) 12, IoT devices 30, and other devices within environment 28 may be configured to communicate with network 16, e.g., with HMS 22, via a cellular base station 36 and a cellular network.

In some examples, computing device(s) 12 and/or computing system 20 may implement one or more algorithms to evaluate the sensed physiological data received from IMDs 10. In some examples, computing device(s) 12 and/or computing system 20 may have greater processing capacity than IMDs 10, enabling more complex analysis of the data. In some examples, the computing device(s) 12 and/or HMS 22 may apply the data to a machine learning model or other artificial intelligence developed algorithm, e.g., to determine whether the data is sufficiently indicative of the degree of heart recovery.

In some examples, HMS 22 may be configured to transmit messages to one or computing devices 38 associated with one or more clinicians 40 via network 16. Care providers may include emergency medical systems (EMS) and hospitals, and may include particular departments within a hospital, such as an emergency department, catheterization lab, cardiology, or a stroke response department. Clinician computing devices 38 may include smartphones, desktop, laptop, or tablet computers, or workstations associated with such systems or entities, or employees of such systems or entities. The messages may include any of the data collected by IMDs 10, computing device(s) 12, and IoT device(s) 30, including sensed patient metrics, time of the degree of heart recovery, location of patient 4, and results of the analysis by IMDs 10, computing device(s) 12, IoT device(s) 30, and/or HMS 22. The information transmitted from HMS 22 to clinicians 40 may improve the timeliness to determine the effectiveness of intervention of HF of patient 4 by clinicians 40, which may improve treatment of patient 4 diagnosed with HF by clinicians 40.

FIG. 2 is a block diagram illustrating an example configuration of IMD 10 of FIG. 1. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may measure impedance, e.g., of tissue proximate to IMD 10, via electrodes 56. The measured impedance may vary based on respiration, cardiac pulse or flow, and a degree of perfusion or edema. Processing circuitry 50 may determine patient metrics relating to respiration, fluid retention, cardiac pulse or flow, perfusion, and/or edema based on the measured impedance.

Sensing circuitry 54 may also monitor signals from electrodes 56 in order to, for example, monitor electrical activity of a heart of patient 4 and produce sensor data for patient 4. In some examples, processing circuitry 50 may identify features of the sensed ECG, such as heart rate, heart rate variability, T-wave alternans, intra-beat intervals (e.g., QT intervals), and/or ECG morphologic features, to detect an episode of cardiac arrhythmia of patient 4.

In some examples, IMD 10 includes one or more sensors 58, such as one or more accelerometers, gyroscopes, microphones, optical sensors, temperature sensors, pressure sensors, and/or chemical sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52. Patient parameters determined from signals from sensors 58 may include intravascular fluid level, interstitial fluid level, oxygen saturation, glucose level, stress hormone level, heart sounds, body motion, activity intensity, sleep duration, sleep quality, body posture, or blood pressure.

Memory 52 may store applications 70 executable by processing circuitry 50, and data 80. Applications 70 may include a recovery surveillance application 72. Processing circuitry 50 may execute recovery surveillance application 72 to generate a degree of patient metric recovery and/or a degree of heart recovery based on combination of one or more of the patient metrics from IMD 10, described herein, which may be stored as sensed data 82. In some examples, sensed data 82 may additionally include patient metrics sensed by other devices, e.g., computing device(s) 12 or IoT device(s) 30, and received via communication circuitry 60. Recovery surveillance application 72 may be configured with a rules engine 74. Rules engine 74 may apply rules 84 to sensed data 82. Rules 84 may include one or more models, algorithms, decision trees, and/or thresholds. In some cases, rules 84 may be developed based on machine learning, e.g., may include one or more machine learning models.

When recovery surveillance application 72 generates a degree of patient metric recovery and/or a degree of heart recovery, recovery surveillance application 72 may store the sensed data 82 that lead to the detection as event data 86. In some examples, in response to generation of a degree of patient metric recovery and/or a degree of heart recovery, processing circuitry 50 transmits, via communication circuitry 60, event data 86 for the event to computing device(s) 12 (FIG. 1). This transmission may be included in a message indicating the degree of patient metric recovery and/or the degree of heart recovery, as described herein. Transmission of the message may occur on an ad hoc basis and as quickly as possible. Communication circuitry 60 may include any suitable hardware, firmware, software, or any combination thereof for wirelessly communicating with another device, such as computing device(s) 12 and/or IoT devices 30.

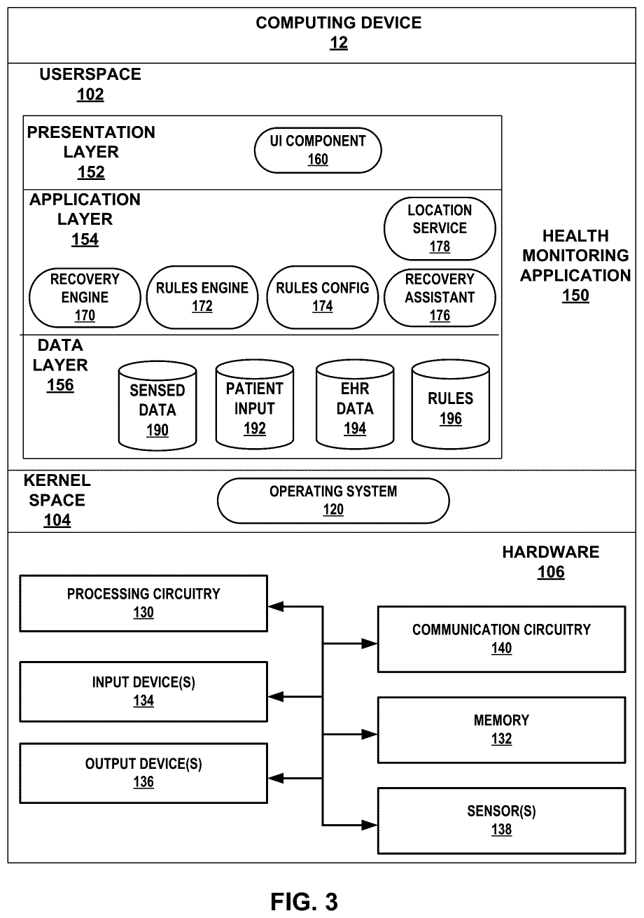

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, IoT devices 30 and/or computing devices 38 and 42 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 132 includes cloud-associated storage.

One or more input devices 134 of computing device 12 may receive input, e.g., from patient 4, clinician 40, or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, haptic, audio, and visual output. Output devices 134 of computing device 12 may include a presence-sensitive screen, sound card, video graphics adapter card, speaker, cathode ray tube monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, accelerometers (e.g., 3-axis accelerometers), an optical sensor, impedance sensors, temperature sensors, pressure sensors, heart sound sensors (e.g., microphones or accelerometers), and other sensors, and sensing circuitry (e.g., including an ADC), similar to those described above with respect to IMDs 10 and FIG. 2.

Communication circuitry 140 of computing device 12 may communicate with other devices by transmitting and receiving data. Communication circuitry 140 may receive data from IMD 10, such as patients metrics and/or higher resolution diagnostic information, from communication circuitry in IMD 10. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Sensed data 190 may include data received from IMD 10, such as patient metrics and/or higher resolution diagnostic information. Application layer 154 may include, but is not limited to, recovery engine 170, rules engine 172, rules configuration component 174, recovery assistant 176, and location service 178. Recovery engine 170 may be responsive to receipt of a transmission from IMD 10 indicating that sensed data 190 from IMD 10 has been received and begin one or more of generating higher resolution diagnostic information, a degree of recovery for one or more patient metrics, and/or a degree of heart recovery of patient 4. Recovery engine 170 may also control performance of any of the operations in response to generation of a degree of recovery for one or more patient metrics and/or a degree of heart recovery ascribed herein to computing device 12, such as transmitting messages to HMS 22 and controlling IoT devices 30.

Rules engine 174 analyzes sensed data 190, and in some examples, patient input 192 and/or EHR data 194, to generate a degree of recovery for one or more patient metrics and/or a degree of heart recovery of patient 4. Sensed data 190 may include data received from IMD 10, such as patient metrics, as part of the transmission, additional data transmitted from IMD 10, e.g., in “real-time,” and physiological and other data related to the condition of patient 4 collected by, for example, computing device(s) 12 and/or IoT devices 30. Sensed data 190 from IMD 10 may further include generated higher resolution diagnostic information. Sensed data 190 may further include additional sensed data than received from IMDs 10. As examples sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include responses to queries posed by health monitoring application 150 regarding the condition of patient 4, input by patient 4 or another user. The queries and responses may occur responsive to the generated degree of patient metric recovery generated degree of heart recovery or may have occurred prior to the generation, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of SCA, tachyarrhythmia, myocardial infarction, stroke, seizure, one or more disease states, such as status of HF, degree of heart recovery after intervention, COPD, renal dysfunction, or hypertension, aspects of disease state, such as ECG characteristics, cardiac ischemia, oxygen saturation, lung fluid, activity, or metabolite level, genetic conditions, congenital anomalies, history of procedures, such as ablation or cardioversion, and healthcare utilization. EHR data 194 may also include cardiac indicators, such as ejection fraction and left-ventricular wall thickness. EHR data 194 may also include demographic and other information of patient 4, such as age, gender, race, height, weight, and BMI.

Rules engine 172 may apply rules 196 to the data. Rules 196 may include one or more models, algorithms, decision trees, and/or thresholds. The rules 196 may include any of the rules discussed above with respect to a relationship of higher resolution diagnostic information to generating a degree of patient metric recovery and/or a degree of heart recovery. The rules 196 may further include additional parameters measured by any of the sensors discussed above.

In some cases, rules 196 may be developed based on machine learning, e.g., may include one or more machine learning models. In some examples, rules 196 and the operation of rules engine 172 may provide a more complex analysis the patient parameter data, e.g., the data received from IMDs 10, than is provided by rules engine 74 and rules 84. In examples in which rules 196 include one or more machine learning models, rules engine 172 may apply feature vectors derived from the data to the model(s).

Rules configuration component 174 may be configured to modify rules 196 (and in some examples rules 84) based on feedback indicating whether the degree of patient metric recovery and/or the degree of heart recovery by IMD 10 and/or computing device 12 were accurate. The feedback may be received from patient 4, or from clinicians 40 and/or EHR 24 via HMS 22. In some examples, rules configuration component 174 may utilize the data sets from true and false detections and confirmations for supervised machine learning to further train models included as part of rules 196.

Rules configuration component 174, or another component executed by processing circuitry of system 2, may select a configuration of rules 196 based on etiological data for patient, e.g., any combination of one or more of the examples of sensed data 190, patient input 192, and EHR data 194 discussed above. In some examples, different sets of rules 196 tailored to different cohorts of patients may be available for selection for patient 4 based on such etiological data.

As discussed above, recovery assistant 176 may provide a conversational interface for patient 4 to exchange information with computing device 12. Responses from the user may be included as patient input 192. Recovery assistant 176 may use natural language processing and context data to interpret utterances by the user. In some examples, in addition to receiving responses to queries posed by the assistant, recovery assistant 176 may be configured to respond to queries posed by the user. In some examples, recovery assistant 176 may provide directions to and respond to queries regarding treatment of patient 4.

Location service 178 may determine the location of computing device 12 and, thereby, the presumed location of patient 4. Location service 178 may use global position system (GPS) data, multilateration, and/or any other known techniques for locating computing devices.

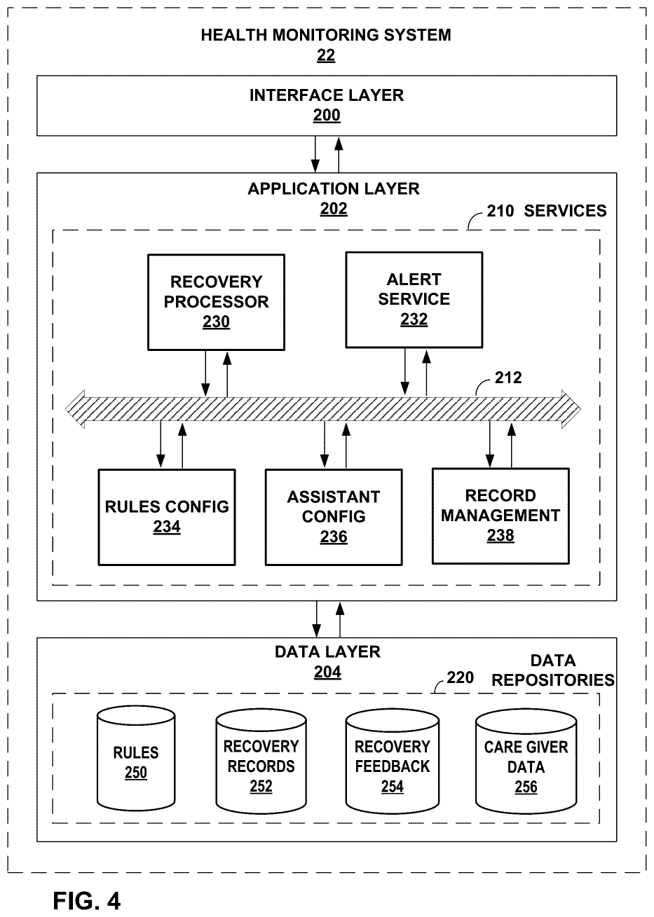

FIG. 4 is a block diagram illustrating an operating perspective of HMS 22. HMS 22 may be implemented in a computing system 20, which may include hardware components such as those of computing device 12, e.g., processing circuitry, memory, and communication circuitry, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing device(s) 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., sensed data from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Recovery processor service 230 may be responsive to receipt of patient metrics, higher resolution diagnostic information, a degree of recovery for one or more patient metrics, and/or a degree of heart recovery from computing device(s) 12 and/or IoT device(s) 30 indicating IMD 10 sensed and transmitted patient metrics and, in some examples, generated higher resolution diagnostic information, a degree of recovery for one or more patient metrics, and/or a degree of heart recovery of patient 4. Recovery processor service 230 may initiate performance of any of the operations in response to receipt of patient metrics, higher resolution diagnostic information, a degree of recovery for one or more patient metrics, and/or a degree of heart recovery ascribed herein to HMS 22, such as analyzing data to generate a degree of recovery for one or more patient metrics, and/or a degree of heart recovery of patient 4 and, in some cases, communicating with patient 4 and clinicians 40.

Record management service 238 may store the patient data included in a received alert message within recovery records 252. Alert service 232 may package the some or all of the data from the recovery record, in some cases with additional information as described herein, into one or more alert messages for transmission to clinicians 40. Care giver data 256 may store data used by alert service 232 to identify to whom to send alerts based on applicability of the care provided by clinicians 40 to the degree of recovery for one or more patient metrics and/or the degree of heart recovery.

In examples in which HMS 22 generates a degree of recovery for one or more patient metrics and/or a degree of heart recovery of patient 4, recovery processor service 230 may apply one or more rules 250 to the patient metrics and/or higher resolution diagnostic information received in the message, e.g., to feature vectors derived by recovery processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to generates a degree of recovery for one or more patient metrics and/or a degree of heart recovery, rules 250 maintained by HMS 22 may include rules 196 utilized by computing device(s) 12 and rules 84 used by IMD 10. In such examples, rules configuration service 250 may be configured to develop and maintain rules 196 and rules 84. Rules configuration service 234 may be configured to develop different sets of rules 84, 196, 250, e.g., different machine learning models, for different cohorts of patients. Rules configuration service 234 may be configured to modify these rules based on recovery feedback data 254 that indicates whether the generations of degrees of patient metric recovery and/or heart recovery by IMD 10, computing device(s) 12, and/or HMS 22 were accurate. Recovery feedback 254 may be received from patient 4, e.g., via computing device(s) 12, or from clinicians 40 and/or EHR 24. In some examples, rules configuration service 234 may utilize recovery records from true and false detections (as indicated by recovery feedback data 254) and confirmations for supervised machine learning to further train models included as part of rules 250.

As illustrated in the example of FIG. 4, services 210 may also include an assistant configuration service 236 for configuring and interacting with recovery assistant 176 implemented in computing device(s) 12 or other computing devices. For example, assistant configuration service 236 may provide recovery assistants updates to their natural language processing and context analyses to improve their operation over time. In some examples, assistant configuration service 236 may apply machine learning techniques to analyze sensed data and recovery assistant interactions stored in recovery records 252, as well as the ultimate disposition of the degree of recovery, e.g., indicated by EHR 24, to modify the operation of recovery assistants, e.g., for patient 4, a class of patients, all patients, or for particular users or devices, e.g., care givers, etc.

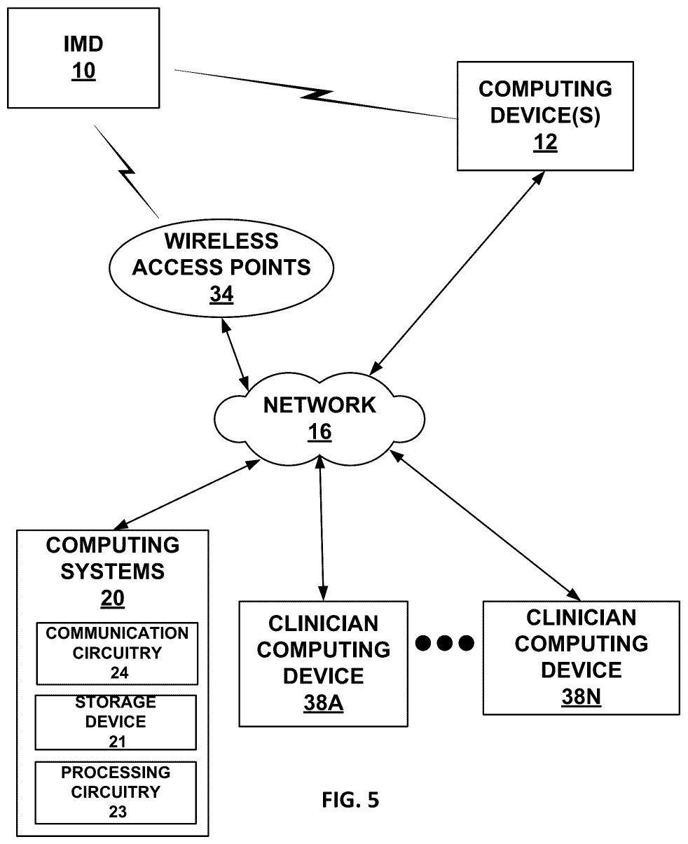

FIG. 5 is a block diagram illustrating an example system that includes wireless access points 34, a network 16, external computing devices, such as computing systems 20, and one or more other clinician computing devices 38A-38N (collectively, “clinician computing devices 38”), which may be coupled to IMD 10 and computing device(s) 12 via network 16, in accordance with one or more techniques described herein. In this example, IMD 10 may use communication circuitry 60 to communicate with computing device(s) 12 via a first wireless connection, and to communicate with wireless access points 34 via a second wireless connection. In the example of FIG. 5, wireless access points 34, computing device(s) 12, computing systems 20, and clinician computing devices 38 are interconnected and may communicate with each other through network 16. Network 16 may include a local area network, wide area network, or global network, such as the Internet. The system of FIG. 5 may be implemented, in some aspects, with general network technology and functionality similar to that provided by the Medtronic CareLink® Network.

Wireless access points 34 may include a device that connects to network 16 via any of a variety of connections, such as telephone dial-up, digital subscriber line (DSL), or cable modem connections. In other examples, wireless access points 34 may be coupled to network 16 through different forms of connections, including wired or wireless connections. In some examples, wireless access points 34 may be a user device, such as a tablet or smartphone, that may be co-located with the patient. IMD 10 may be configured to transmit data, such as impedance value information, impedance scores, and/or cardiac electrograms (EGMs), to wireless access points 34. Wireless access points 34 may then communicate the retrieved data to computing systems 20 via network 16.

In some cases, computing systems 20 may be configured to provide a secure storage site for data that has been collected from IMD 10 and/or computing device(s) 12. In some cases, computing systems 20 may assemble data in web pages or other documents for viewing by trained professionals, such as clinicians, via clinician computing devices 38. One or more aspects of the illustrated system of FIG. 5 may be implemented with general network technology and functionality, which may be similar to that provided by the Medtronic CareLink® Network.

In some examples, computing systems 20 may monitor patient metrics received from IMD 10 and/or computing device(s) 12 via network 16, to generate a degree of patient metric recovery and/or a degree of heart recovery of patient 4 using any of the techniques described herein. Computing systems 20 may provide alerts via network 16 to patient 4 via wireless access points 34, or to one or more clinicians via computing devices 100. In examples such as those described above in which IMD 10 and/or computing device(s) 12 monitor the patient metrics, computing systems 20 may receive an alert from IMD 10 or computing device(s) 12 via network 16, and provide alerts to one or more clinicians via clinician computing devices 38. In some examples, computing systems 20 may generate web-pages to provide alerts and information regarding the patient metrics, higher resolution diagnostic information, degree of patient metric recovery, and/or degree of heart recovery, and may include a memory to store alerts, patient metrics, higher resolution diagnostic information, degrees of patient metric recovery, and/or degrees of heart recovery for a plurality of patients.

In some examples, one or more of clinician computing devices 38 may be a tablet or other smart device located with a clinician, by which the clinician may program, receive alerts from, and/or interrogate IMD 10. For example, the clinician may access data collected by IMD 10 through a clinician computing device 38, such as when patient 4 is in between clinician visits, to check on a degree of recovery for one or more patient metrics and/or a degree of heart recovery after an intervention. In some examples, the clinician may enter instructions for a medical intervention for patient 4 into an application executed by clinician computing device 38, such as based on a degree of recovery determined by IMD 10, computing device(s) 12, computing systems 20, or any combination thereof, or based on other patient data known to the clinician. Clinician computing device 100 then may transmit the instructions for medical intervention to another of clinician computing devices 100 located with patient 4 or a caregiver of patient 4.

In some examples, instructions for medical intervention may include an instruction to change a drug dosage, timing, or selection, to schedule a visit with the clinician, or to seek medical attention. In further examples, a clinician computing device 38 may generate an alert to patient 4 based on a degree of recovery of patient 4, which may enable patient 4 to proactively seek medical attention prior to receiving instructions for a medical intervention. In this manner, patient 4 may be empowered to take action, as needed, to address his or her medical status, which may help improve clinical outcomes for patient 4.

In the example illustrated by FIG. 5, computing system 20 includes a storage device 21, e.g., to store data retrieved from IMD 10, processing circuitry 23, and communication circuitry. Although not illustrated in FIG. 5 clinician computing devices 38 may similarly include a storage device and processing circuitry. Processing circuitry 23 may include one or more processors that are configured to implement functionality and/or process instructions for execution within computing systems 20. For example, processing circuitry 23 may be capable of processing instructions stored in storage device 21. Processing circuitry 23 may include, for example, microprocessors, DSPs, ASICs, FPGAs, or equivalent discrete or integrated logic circuitry, or a combination of any of the foregoing devices or circuitry. Accordingly, processing circuitry 23 may include any suitable structure, whether in hardware, software, firmware, or any combination thereof, to perform the functions ascribed herein to processing circuitry 98. Processing circuitry 23 of computing systems 20 and/or the processing circuitry of clinician computing devices 38 may implement any of the techniques described herein to analyze impedance values received from IMD 10, e.g., to generate a degree of patient metric recovery and/or a degree of heart recovery of patient 4 (e.g., worsening HF). Communication circuitry 24 may include any suitable hardware, firmware, software, or any combination thereof for wirelessly communicating with another device, such as IMD, computing device(s) 12, and/or clinician computing devices 38.

Storage device 21 may include a computer-readable storage medium or computer-readable storage device. In some examples, storage device 21 includes one or more of a short-term memory or a long-term memory. Storage device 21 may include, for example, RAM, DRAM, SRAM, magnetic discs, optical discs, flash memories, or forms of EPROM or EEPROM. In some examples, storage device 21 is used to store data indicative of instructions for execution by processing circuitry 23.

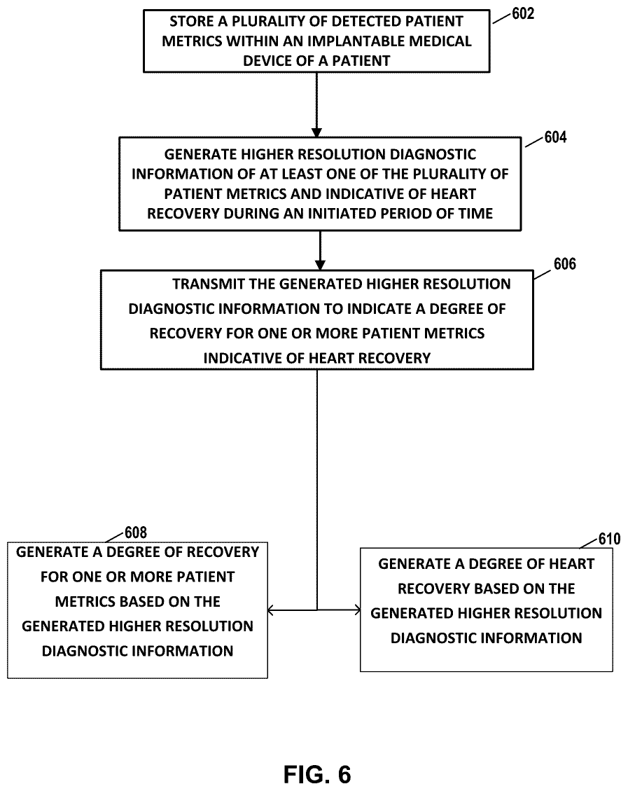

FIG. 6 is a flow diagram illustrating an example operation of transmitting the generated higher resolution diagnostic information to indicate a degree of recovery, generating a degree of recovery for one or more patient metrics based on the generated higher resolution diagnostic information, and/or generating a degree of heart recovery based on the generated higher resolution diagnostic information in accordance with one or more techniques of this disclosure. The example operation of FIG. 6 may be performed by processing circuitry and communication circuitry of any one of IMD 10, computing device(s) 12, computing systems 20, clinician computing devices 38, or IoT devices 30 (e.g., by processing circuitry 50, 130, or 23, communication circuitry 60, 140, 24), or by processing circuitry and/or communication circuitry of two or more of these devices respectively performing portions of the example operation.

As seen in the example of FIG. 6, processing circuitry initially may store a plurality of automatically detected patient metrics within an implantable medical device of a patient (602). Next, a processing circuitry may generate higher resolution diagnostic information of the plurality of patient metrics and indicative of heart recovery of the patient during an initiated period of time. The initiated period of time may be initiated based on one or more of an intervention being provided to the patient and receiving a user input (604). Next, communication circuitry may transmit the generated higher resolution diagnostic information to indicate a degree of recovery for one or more patient metrics indicative of heart recovery (606).

As seen in the example, the processing circuitry may generate a degree of recovery for one or more patient metrics based on the generated higher resolution diagnostic information. (608). The processing may also generate a degree of heart recovery based on the generated higher resolution diagnostic information (610).

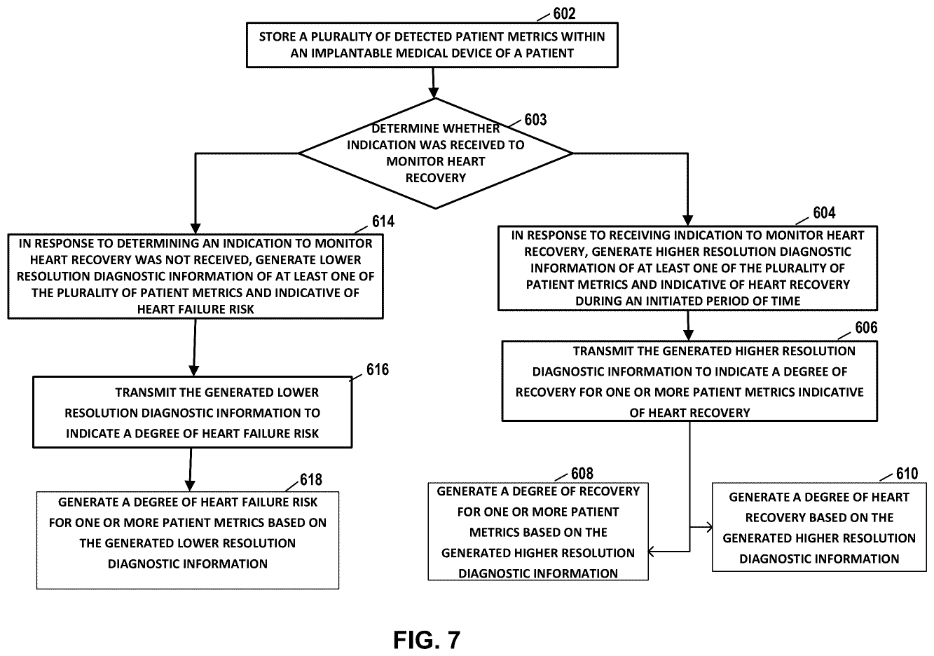

FIG. 7 is a flow diagram illustrating an example operation similar to FIG. 6. The example operation of FIG. 7 may be performed by processing circuitry and communication circuitry of any one of IMD 10, computing device(s) 12, computing systems 20, clinician computing devices 38, or IoT devices 30 (e.g., by processing circuitry 50, 130, or 23, communication circuitry 60, 140, 24), or by processing circuitry and/or communication circuitry of two or more of these devices respectively performing portions of the example operation.

As seen in the example of FIG. 7, processing circuitry initially may store a plurality of automatically detected patient metrics within an implantable medical device of a patient (602). Next, a processing circuitry may determine whether an indication was received to monitor heart recovery (603). In some examples, an indication may be that an intervention was provided to the patient. In some examples, an indication may be a user input, such as by a clinician or a patient.

In response to a processing circuitry receiving an indication to monitor heart recovery, a processing circuitry may generate higher resolution diagnostic information of the plurality of patient metrics and indicative of heart recovery of the patient during an initiated period of time. The initiated period of time may be initiated based on one or more of an intervention being provided to the patient and receiving a user input (604). In addition, in response to a processing circuitry receiving an indication to monitor heart recovery, the flow chart continues as shown in FIG. 6.

In response to a processing circuitry determining an indication to monitor heart recovery was not received, a processing circuitry may generate lower resolution diagnostic information of the plurality of patient metrics and indicative of HF risk (614). Next, communication circuitry may transmit the generated lower resolution diagnostic information to indicate a degree of HF risk for one or more patient metrics indicative of HF risk. (616). As seen in the example, the processing circuitry may generate a degree of HF risk for one or more patient metrics based on the generated lower resolution diagnostic information. (618).

In some examples, the processing circuitry may compare each of the plurality of detected patient metrics detected at an end of the initiated period of time to a respective detected patient metric detected at a beginning of the initiated period of time, and automatically generate a degree of heart recovery based on the comparison, wherein the degree of heart recovery is indicative of an amount the heart recovered over the initiated period of time. In some examples, a length of the period of time may be based on a type of detected patient metric being compared.

In some examples, the processing circuitry may generate an average of each of the plurality of detected patient metrics detected over the initiated period of time, compare each of the averages to a respective detected patient metric detected at a beginning of the initiated period of time, and automatically generate a degree of heart recovery based on the comparison. The degree of heart recovery indicating an amount the heart recovered over the period of time.

In some examples, the processing circuitry may generate an average of each of the plurality of detected patient metrics detected over a second period of time, the second period of time beginning after the initiated period of time is initiated, compare each of the averages to a respective detected patient metric detected at a beginning of the period of time, and automatically generate a degree of heart recovery based on the comparison. The degree of heart recovery indicating an amount the heart recovered over the period of time.

In some examples, the processing circuitry may generate a rate of change of each of the plurality of detected patient metrics detected over the initiated period of time, compare each of the rates of change to a respective threshold level, and automatically generate a degree of patient metric recovery of each of the plurality of detected patient metrics based on the comparison. The degree of patient metric recovery indicating an amount each parameter recovered over the initiated period of time. The processing circuitry may further automatically generate a degree of heart recovery based on the generated degrees of patient metric recovery. The degree of heart recovery indicating an amount the heart recovered or worsened over the initiated period of time. In some examples, the processing circuitry may generate a degree of heart recovery with a probability model based on the plurality of generated patient metrics.

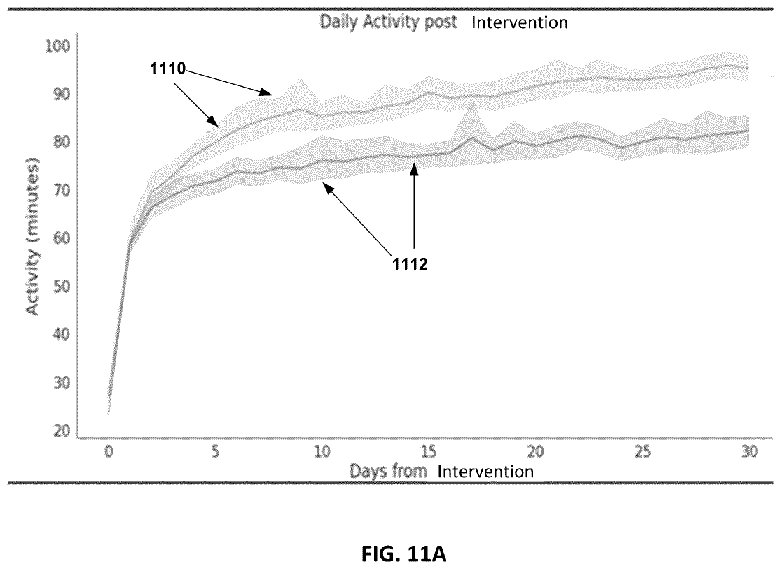

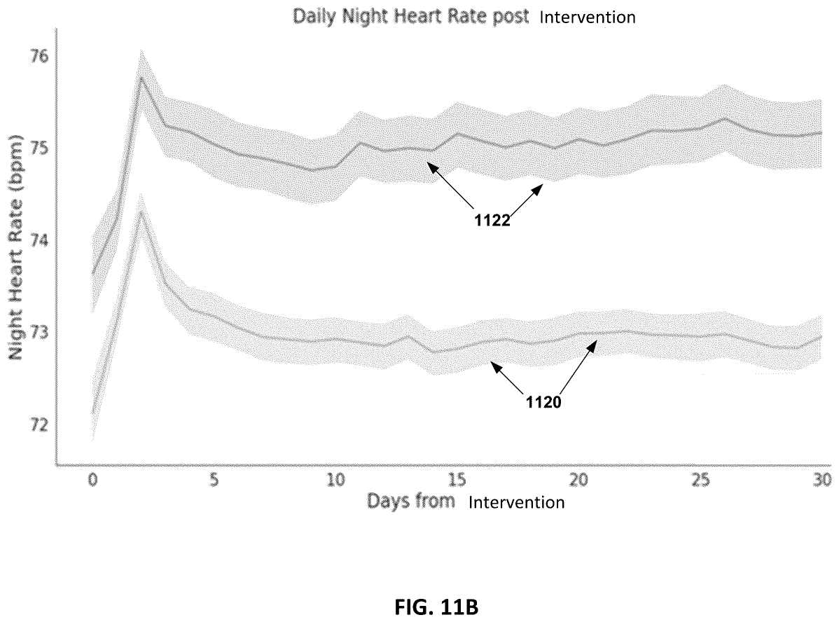

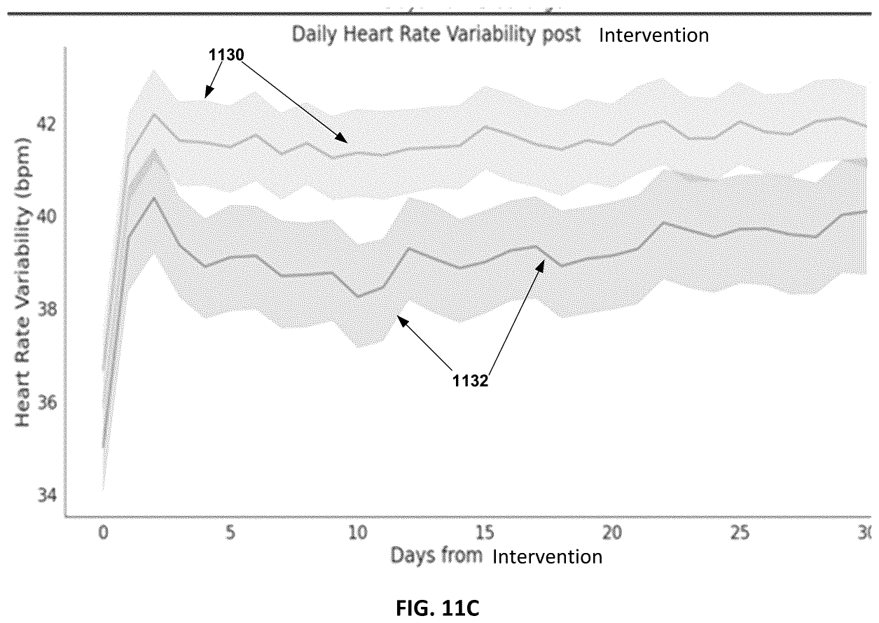

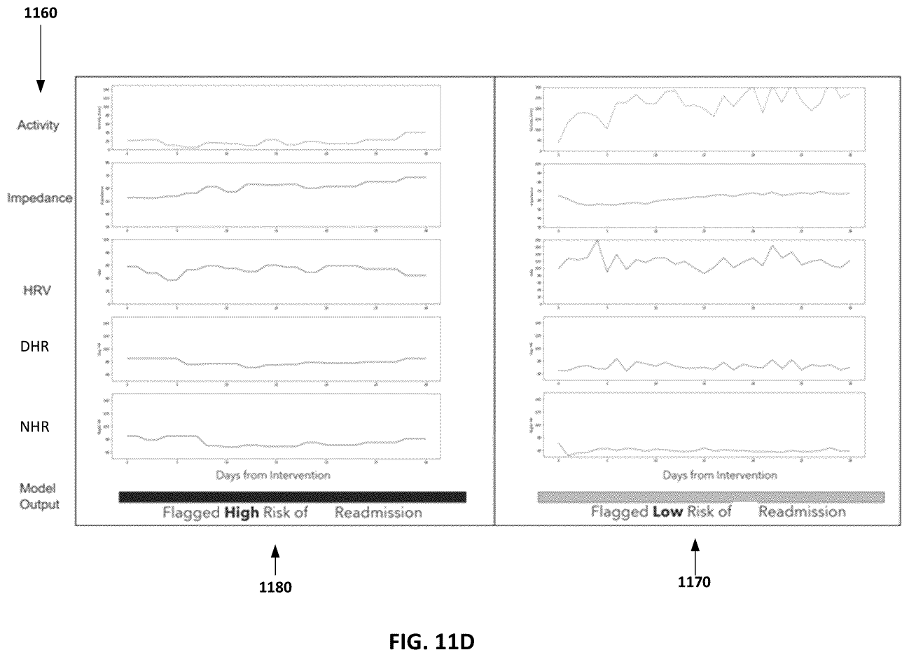

FIGS. 11A-11C illustrate some examples of patient metric recovery over a period of time and generating a degree of heart recovery based on the respective patient metric recovery. FIG. 11A is a graph showing daily activity of a patient versus days from an intervention provided to the patient. In some examples, patient activity may be determined in minutes of activity above an activity threshold per day, but patient activity may be determined using other values, such as intensity of patient activity per day or other time period.

FIG. 11A shows an example of representative data showing an amount of patient daily activity with respect to time from intervention that indicates whether the intervention was an effective intervention 1110 or an ineffective intervention 1112. In some examples, patient daily activity indicating an effective intervention 1110 may include patient daily activity minutes being above 74 minutes at 5 days from intervention but being over 80 minutes 10 days from intervention. In some examples, patient daily activity indicating an effective intervention 1110 may include patient daily activity minutes being between two amounts at a particular day after invention, such as between 80 and 90 minutes 10 days after intervention. In some examples, patient daily activity indicating an ineffective intervention 1112 may include patient daily activity minutes being below 72 minutes at 5 days from intervention, but being below 78 minutes 10 days from intervention. In some examples, patient daily activity indicating an ineffective intervention 1112 may include patient daily activity minutes being between two amounts at a particular day after invention, such as between 65 and 78 minutes 10 days after intervention.

In some examples, processing circuitry may determine an effective intervention threshold and/or an ineffective intervention threshold of patient daily activity data based on representative data showing an amount of patient daily activity with respect to time from intervention that indicates whether the intervention was an effective intervention 1110 or an ineffective intervention 1112, such as shown in FIG. 11A. In some examples, processing circuitry may determine patient daily activity satisfying an effective intervention threshold indicates the patient heart is recovering satisfactorily and additional intervention, such as hospital readmission, may not be necessary. In some examples, processing circuitry may determine patient daily activity satisfying an ineffective intervention threshold indicates the patient heart is not recovering satisfactorily and additional intervention, such as hospital readmission, may be necessary. In some examples, processing circuitry may generate a degree of heart recovery based, at least in part, on comparing patient daily activity minutes with respect to at least one of effective intervention threshold and/or ineffective intervention threshold over a period of time after intervention, such as up to 5 days, 7 days, 10 days, 15 days, 25 days, 30 days, or any other amount of time of interest after the beginning of the initiated period.

In some examples, processing circuitry may train a ML model based on daily activity data with respect to time from intervention that indicates whether the intervention was an effective intervention 1110 or an ineffective intervention 1112, such as shown in FIG. 11A, to determine/predict whether an intervention may be effective, such as determining/predicting a likelihood of rehospitalization after an initial hospitalization. In some examples, processing circuitry may apply data of patient activity and degree of heart recovery after intervention of a plurality of patients to a ML model to determine one or more of effective intervention threshold or ineffective intervention threshold.

FIG. 11B shows an example of representative data showing daily night heart rate of a patient versus days from an intervention provided to the patient. In some examples, daily night heart rate may be a mean, median, or mode of sensed or determined heart rate during a period of rest/sleep and/or during a period of time at night (e.g., 10:00 pm to 6:00 am). In some examples, as shown in FIG. 11B, patient daily night heart rate data may indicate whether an intervention was an effective intervention 1120 or an ineffective intervention 1122. In some examples, patient daily night heart rate indicating an effective intervention 1120 may include patient daily night heart rate being below 73.5 beats per minute (bpm) at 5 days from intervention, but being below 73 bpm 10 days from intervention. In some examples, patient daily night heart rate indicating an effective intervention 1120 may include patient daily night heart rate being between two amounts at a particular day after invention, such as between 72.8 and 73.2 bpm 10 days after intervention. In some examples, patient daily night heart rate indicating an ineffective intervention 1122 may include patient daily night heart rate being above 74.8 bpm at 5 days from intervention, but being above 74.5 bpm 10 days from intervention. In some examples, patient daily night heart rate indicating an ineffective intervention 1122 may include patient daily night heart rate being between two amounts at a particular day after invention, such as between 74.5 and 75.2 bpm 10 days after intervention.

In some examples, processing circuitry may determine an effective intervention threshold and/or an ineffective intervention threshold of patient daily night heart rate data based on representative data showing daily night heart rate of a patient versus time from an intervention provided to the patient that indicates whether the intervention was an effective intervention 1120 or an ineffective intervention 1122, such as shown in FIG. 11B. In some examples, processing circuitry may determine daily night heart rate satisfying an effective intervention threshold indicates the patient heart is recovering satisfactorily and additional intervention, such as hospital readmission, may not be necessary. In some examples, processing circuitry may determine daily night heart rate satisfying an ineffective intervention threshold indicates the patient heart is not recovering satisfactorily and additional intervention, such as hospital readmission, may be necessary. In some examples, processing circuitry may generate a degree of heart recovery based, at least in part, on comparing patient daily night heart rate with respect to at least one of effective intervention threshold and/or ineffective intervention threshold over a period of time after intervention, such as up to 5 days, 7 days, 10 days, 15 days, 25 days, 30 days, or any other amount of time of interest after the beginning of the initiated period.

In some examples, processing circuitry may train a ML model based on daily night heart rate data with respect to time from intervention that indicates whether the intervention was an effective intervention 1120 or an ineffective intervention 1122, such as shown in FIG. 11B, to determine/predict whether an intervention may be effective, such as determining/predicting a likelihood of rehospitalization after an initial hospitalization. In some examples, processing circuitry may apply data of daily night heart rate and degree of heart recovery after intervention of a plurality of patients to a ML model to determine one or more of effective intervention threshold or ineffective intervention threshold.

FIG. 11C shows an example of representative data showing daily heart rate variability of a patient versus days from an intervention was provided to the patient. In some examples, as shown in FIG. 11C, patient daily heart rate variability may indicate whether an intervention was an effective intervention 1130 or an ineffective intervention 1132. In some examples, daily heart rate variability indicating an effective intervention 1130 may include patient daily heart rate variability being above 40.5 bpm at 5 days from intervention, but being above 40 bpm 10 days from intervention. In some examples, daily heart rate variability indicating an effective intervention 1130 may include patient daily heart rate variability being between two amounts at a particular day after invention, such as between 40 and 42 bpm 10 days after intervention. In some examples, daily heart rate variability indicating an ineffective intervention 1132 may include patient daily heart rate variability being below 40 bpm at 5 days from intervention, but being below 39.5 bpm 10 days from intervention. In some examples, daily heart rate variability indicating an ineffective intervention 1132 may include patient daily heart rate variability being between two amounts at a particular day after invention, such as between 37.5 and 39.5 bpm 10 days after intervention.

In some examples, processing circuitry may determine an effective intervention threshold and/or an ineffective intervention threshold of patient daily heart rate variability based on representative data showing patient daily heart rate variability with respect to time from intervention that indicates whether the intervention was an effective intervention 1130 or an ineffective intervention 1132, such as shown in FIG. 11C.

In some examples, processing circuitry may determine patient daily heart rate variability satisfying an effective intervention threshold indicates the patient heart is recovering satisfactorily and additional intervention, such as hospital readmission, may not be necessary. In some examples, processing circuitry may determine patient daily heart rate variability satisfying an ineffective intervention threshold indicates the patient heart is not recovering satisfactorily and additional intervention, such as hospital readmission, may be necessary. In some examples, processing circuitry may generate a degree of heart recovery based, at least in part, on comparing patient daily heart rate variability with respect to at least one of effective intervention threshold and/or ineffective intervention threshold over a period of time after intervention, such as up to 5 days, 7 days, 10 days, 15 days, 25 days, or 30 days.

In some examples, processing circuitry may train a ML model based on daily heart rate variability data with respect to time from intervention that indicates whether the intervention was an effective intervention 1130 or an ineffective intervention 1132, such as shown in FIG. 11C, to determine/predict whether an intervention may be effective, such as determining/predicting a likelihood of rehospitalization after an initial hospitalization. In some examples, processing circuitry may apply data of daily heart rate variability and degree of heart recovery after intervention of a plurality of patients to a ML model to determine one or more of effective intervention threshold or ineffective intervention threshold.

FIG. 11D shows an example of representative data showing various patient data 1160, the patient data 1160 may include one or more of activity, impedance, heart rate variability (HRV), daily heart rate (DHR), and/or night heart rate (NHR) of a patient versus days from an intervention was provided to the patient.

In some examples, processing circuitry may determine an effective intervention threshold and/or an ineffective intervention threshold of patient data 1160 based on representative data showing patient data with respect to time from intervention that indicates whether the intervention was an effective intervention that indicates a patient is at low risk of hospital readmission 1170 or an ineffective intervention that indicates a patient is at high risk of hospital readmission 1180, such as shown in FIG. 11D.

In some examples, processing circuitry may determine patient data 1160 satisfying an effective intervention threshold indicates the patient heart is recovering satisfactorily and additional intervention, such as hospital readmission, may not be necessary 1170. In some examples, processing circuitry may determine patient data 1160 satisfying an ineffective intervention threshold indicates the patient heart is not recovering satisfactorily and additional intervention, such as hospital readmission, may be necessary 1180. In some examples, processing circuitry may generate a degree of heart recovery based, at least in part, on comparing patient data 1160 with respect to at least one of effective intervention threshold and/or ineffective intervention threshold over a period of time after intervention, such as up to 5 days, 7 days, 10 days, 15 days, 25 days, or 30 days.

In some examples, processing circuitry may train a ML model based on patient data including one or more of activity, impedance, HRV, DHR, and/or NHR with respect to time from intervention that indicates whether the intervention was an effective intervention or an ineffective intervention, such as shown in FIG. 11D, to determine/predict whether an intervention may be effective, such as determining/predicting a likelihood of rehospitalization after an initial hospitalization. In some examples, processing circuitry may apply patient data of one or more of activity, impedance, HRV, DHR, and/or NHR and degree of heart recovery after intervention of a plurality of patients to a ML model to determine one or more of effective intervention threshold or ineffective intervention threshold.

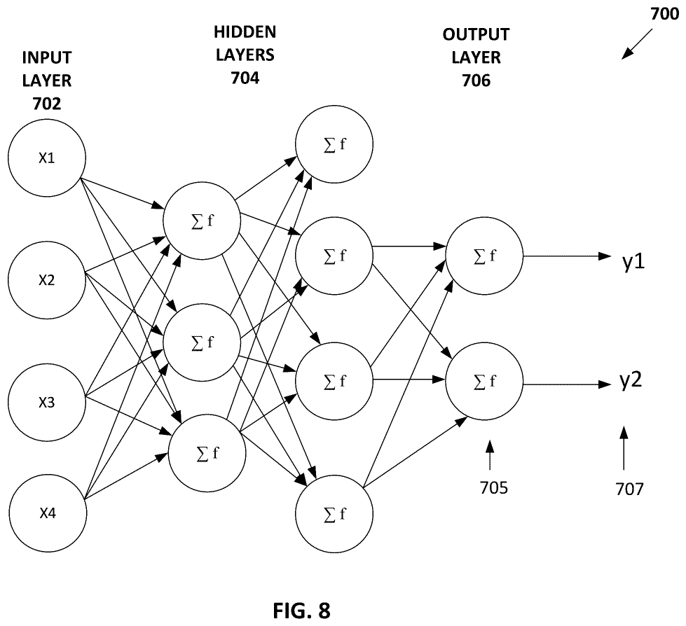

FIG. 8 is a conceptual diagram illustrating an example machine learning model 700 configured to generate one or more values indicative of a degree of recovery from a treatment, e.g., for heart failure or another patient condition, based on physiological parameter values, e.g., sensed by an IMD and/or other devices as described herein. Machine learning model 700 is an example of a deep learning model, or deep learning algorithm. One or more of IMD 10, computing devices 12, or computing system 20 (e.g., rules configuration component 174 and/or 234) may train, store, and/or utilize machine learning model 700, but other devices may apply inputs associated with a particular patient to machine learning model 700 in other examples. Some non-limiting examples of machine learning techniques include Bayesian probability models, Support Vector Machines, K-Nearest Neighbor algorithms, and Multi-layer Perceptron.

As shown in the example of FIG. 8, machine learning model 700 may include three layers. These three layers include input layer 702, hidden layer 704, and output layer 706. Output layer 706 comprises the output from the transfer function 705 of output layer 706. Input layer 702 represents each of the input values X1 through X4 provided to machine learning model 700. The number of inputs may be less than or greater than 4, including much greater than 4, e.g., hundreds or thousands. In some examples, the input values may be any of the of physiological or other patient parameter values described herein.

Each of the input values for each node in the input layer 702 is provided to each node of hidden layer 704. In the example of FIG. 8, hidden layers 704 include two layers, one layer having four nodes and the other layer having three nodes, but fewer or greater number of nodes may be used in other examples. Each input from input layer 702 is multiplied by a weight and then summed at each node of hidden layers 704. During training of machine learning model 700, the weights for each input are adjusted to establish the relationship between input physiological parameter values and one or more output values indicative of a health state of the patient. In some examples, one hidden layer may be incorporated into machine learning model 700, or three or more hidden layers may be incorporated into machine learning model 700, where each layer includes the same or different number of nodes.

The result of each node within hidden layers 704 is applied to the transfer function of output layer 706. The transfer function may be liner or non-linear, depending on the number of layers within machine learning model 700. Example non-linear transfer functions may be a sigmoid function or a rectifier function. The output 707 of the transfer function may be a value or values indicative of recovery of a health condition following intervention, or more generally the state of a health condition of the patient. By applying the patient parameter data to a machine learning model, such as machine learning model 700, processing circuitry of system 2 is able to determine the health condition with great accuracy, specificity, and sensitivity.

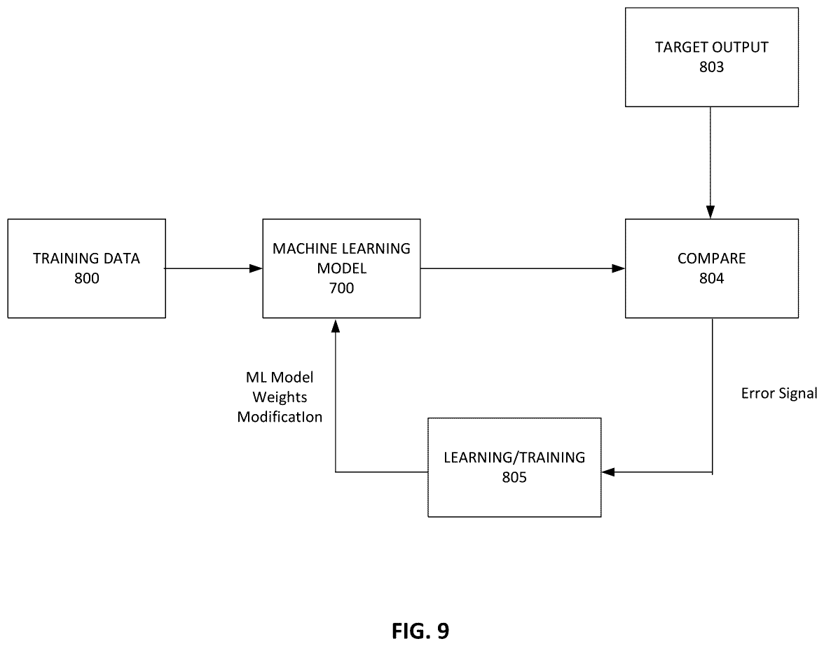

FIG. 9 is an example of a machine learning model 700 being trained using supervised and/or reinforcement learning techniques. Machine learning model 700 may be implemented using any number of models for supervised and/or reinforcement learning, such as but not limited to, an artificial neural network, a decision tree, naïve Bayes network, support vector machine, or k-nearest neighbor model, to name only a few examples. In some examples, processing circuitry one or more of IMD 10, external device 12, and/or computing system 20 (e.g., rules configuration component 174 and/or 234) initially trains the machine learning model 700 based on training set data 800 including numerous instances of input data corresponding to various patient conditions. An output of the machine learning model 700 may be compared 804 to the target output 803, e.g., as determined based on the label. Based on an error signal representing the comparison, the processing circuitry implementing a learning/training function 805 may send or apply a modification to weights of machine learning model 700 or otherwise modify/update the machine learning model 700. For example, one or more of IMD 10, computing device 12, and/or computing system 20 may, for each training instance in the training set 800, modify machine learning model 700 to change a score generated by the machine learning model 700 in response to data applied to the machine learning model 700.

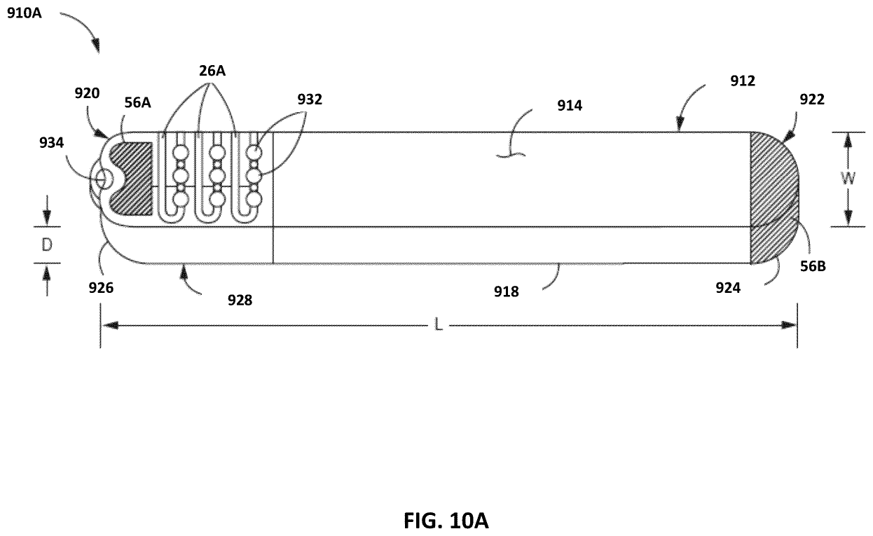

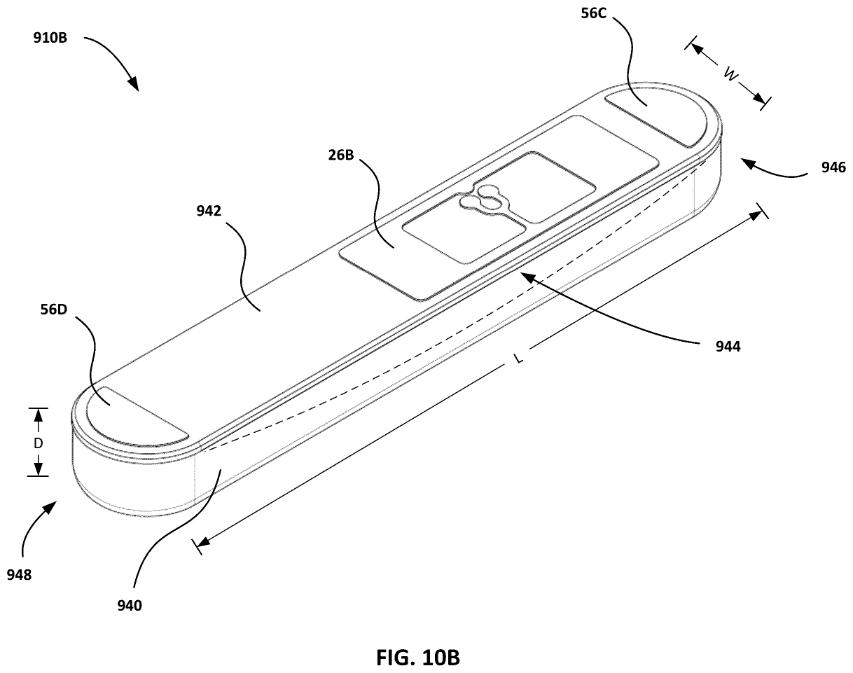

FIG. 10A is a perspective drawing illustrating an IMD 910A, which may be an example configuration of IMD 10 of FIGS. 1 and 2 as an ICM. In the example shown in FIG. 10A, IMD 910A may be embodied as a monitoring device having housing 912, proximal electrode 56A and distal electrode 56B. Housing 912 may further comprise first major surface 914, second major surface 918, proximal end 920, and distal end 922. Housing 912 encloses electronic circuitry located inside the IMD 910A and protects the circuitry contained therein from body fluids. Housing 912 may be hermetically sealed and configured for subcutaneous implantation. Electrical feedthroughs provide electrical connection of electrodes 56A and 56B.

In the example shown in FIG. 10A, IMD 910A is defined by a length L, a width W and thickness or depth D and is in the form of an elongated rectangular prism wherein the length L is much larger than the width W, which in turn is larger than the depth D. In one example, the geometry of the IMD 910A—in particular a width W greater than the depth D—is selected to allow IMD 910A to be inserted under the skin of the patient using a minimally invasive procedure and to remain in the desired orientation during insertion. For example, the device shown in FIG. 10A includes radial asymmetrics (notably, the rectangular shape) along the longitudinal axis that maintains the device in the proper orientation following insertion. For example, the spacing between proximal electrode 56A and distal electrode 56B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 910A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 914 may range from 3 mm to 15, mm, from 3 mm to 10 mm, or from 5 mm to 15 mm, and may be any single or range of widths between 3 mm and 15 mm. The thickness of depth D of IMD 910A may range from 2 mm to 15 mm, from 2 mm to 9 mm, from 2 mm to 5 mm, from 5 mm to 15 mm, and may be any single or range of depths between 2 mm and 15 mm. In addition, IMD 910A according to an example of the present disclosure is has a geometry and size designed for case of implant and patient comfort. Examples of IMD 910A described in this disclosure may have a volume of three cubic centimeters (cm) or less, 1.5 cubic cm or less or any volume between three and 1.5 cubic centimeters.

In the example shown in FIG. 10A, once inserted within the patient, the first major surface 914 faces outward, toward the skin of the patient while the second major surface 918 is located opposite the first major surface 914. In addition, in the example shown in FIG. 10A, proximal end 920 and distal end 922 are rounded to reduce discomfort and irritation to surrounding tissue once inserted under the skin of the patient. IMD 910A, including instrument and method for inserting IMD 910A is described, for example, in U.S. Patent Publication No. 2014/0276928, incorporated herein by reference in its entirety.

Proximal electrode 56A is at or proximate to proximal end 920, and distal electrode 36B is at or proximate to distal end 922. Proximal electrode 56A and distal electrode 56B are used to sense cardiac EGM signals, e.g., ECG signals, and measure interstitial impedance thoracically outside the ribcage, which may be sub-muscularly or subcutaneously. EGM signals and impedance measurements may be stored in a memory of IMD 910A, and data may be transmitted via integrated antenna 26A to another device, which may be another implantable device or an external device, such as computing device 12. In some example, electrodes 56A and 56B may additionally or alternatively be used for sensing any bio-potential signal of interest, which may be, for example, an EGM, EEG, EMG, or a nerve signal, from any implanted location. Housing 912 may house the circuitry of IMD 10 illustrated in FIG. 2.

In the example shown in FIG. 910A, proximal electrode 56A is at or in close proximity to the proximal end 920 and distal electrode 56B is at or in close proximity to distal end 922. In this example, distal electrode 56B is not limited to a flattened, outward facing surface, but may extend from first major surface 914 around rounded edges 924 and/or end surface 926 and onto the second major surface 918 so that the electrode 56B has a three-dimensional curved configuration. In some examples, electrode 56B is an uninsulated portion of a metallic, e.g., titanium, part of housing 912.

In the example shown in FIG. 10A, proximal electrode 56A is located on first major surface 914 and is substantially flat, and outward facing. However, in other examples proximal electrode 56A may utilize the three dimensional curved configuration of distal electrode 56B, providing a three dimensional proximal electrode (not shown in this example). Similarly, in other examples distal electrode 56B may utilize a substantially flat, outward facing electrode located on first major surface 914 similar to that shown with respect to proximal electrode 56A.

The various electrode configurations allow for configurations in which proximal electrode 56A and distal electrode 56B are located on both first major surface 914 and second major surface 918. In other configurations, such as that shown in FIG. 10A, only one of proximal electrode 56A and distal electrode 56B is located on both major surfaces 914 and 918, and in still other configurations both proximal electrode 56A and distal electrode 56B are located on one of the first major surface 914 or the second major surface 918 (e.g., proximal electrode 56A located on first major surface 914 while distal electrode 56B is located on second major surface 918). In another example, IMD 910A may include electrodes on both major surface 914 and 918 at or near the proximal and distal ends of the device, such that a total of four electrodes are included on IMD 910A. Electrodes 56A and 56B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 10A, proximal end 920 includes a header assembly 928 that includes one or more of proximal electrode 56A, integrated antenna 26A, anti-migration projections 932, and/or suture hole 934. Integrated antenna 26A is located on the same major surface (i.e., first major surface 914) as proximal electrode 56A and is also included as part of header assembly 928. Integrated antenna 26A allows IMD 910A to transmit and/or receive data. In other examples, integrated antenna 26A may be formed on the opposite major surface as proximal electrode 56A, or may be incorporated within the housing 912 of IMD 910A. In the example shown in FIG. 10A, anti-migration projections 932 are located adjacent to integrated antenna 26A and protrude away from first major surface 914 to prevent longitudinal movement of the device. In the example shown in FIG. 10A, anti-migration projections 932 include a plurality (e.g., nine) small bumps or protrusions extending away from first major surface 914. As discussed above, in other examples anti-migration projections 932 may be located on the opposite major surface as proximal electrode 56A and/or integrated antenna 26A. In addition, in the example shown in FIG. 10A, header assembly 928 includes suture hole 934, which provides another means of securing IMD 910A to the patient to prevent movement following insertion. In the example shown, suture hole 934 is located adjacent to proximal electrode 56A. In one example, header assembly 928 is a molded header assembly made from a polymeric or plastic material, which may be integrated or separable from the main portion of IMD 910A.

FIG. 10B is a perspective drawing illustrating another IMD 910B, which may be another example configuration of IMD 10 from FIGS. 1 and 2 as an ICM. IMD 910B of FIG. 10B may be configured substantially similarly to IMD 910A of FIG. 10A, with differences between them discussed herein.

IMD 910B may include a leadless, subcutaneously-implantable monitoring device, e.g. an ICM. IMD 910B includes housing having a base 940 and an insulative cover 942. Proximal electrode 56C and distal electrode 56D may be formed or placed on an outer surface of cover 942. Various circuitries and components of IMD 910B, e.g., described with respect to FIG. 3, may be formed or placed on an inner surface of cover 942, or within base 940. In some examples, a battery or other power source of IMD 910B may be included within base 940. In the illustrated example, antenna 26B is formed or placed on the outer surface of cover 942, but may be formed or placed on the inner surface in some examples. In some examples, insulative cover 942 may be positioned over an open base 940 such that base 940 and cover 942 enclose the circuitries and other components and protect them from fluids such as body fluids. The housing including base 940 and insulative cover 942 may be hermetically sealed and configured for subcutaneous implantation.

Circuitries and components may be formed on the inner side of insulative cover 942, such as by using flip-chip technology. Insulative cover 942 may be flipped onto a base 940. When flipped and placed onto base 940, the components of IMD 910B formed on the inner side of insulative cover 942 may be positioned in a gap 944 defined by base 940. Electrodes 56C and 56D and antenna 26B may be electrically connected to circuitry formed on the inner side of insulative cover 942 through one or more vias (not shown) formed through insulative cover 942. Insulative cover 942 may be formed of sapphire (i.e., corundum), glass, parylene, and/or any other suitable insulating material. Base 940 may be formed from titanium or any other suitable material (e.g., a biocompatible material). Electrodes 56C and 56D may be formed from any of stainless steel, titanium, platinum, iridium, or alloys thereof. In addition, electrodes 56C and 56D may be coated with a material such as titanium nitride or fractal titanium nitride, although other suitable materials and coatings for such electrodes may be used.

In the example shown in FIG. 10B, the housing of IMD 910B defines a length L, a width W and thickness or depth D and is in the form of an elongated rectangular prism wherein the length L is much larger than the width W, which in turn is larger than the depth D, similar to IMD 910A of FIG. 10A. For example, the spacing between proximal electrode 56C and distal electrode 56D may range from 5 mm to 50 mm, from 30 mm to 50 mm, from 35 mm to 45 mm, and may be any single spacing or range of spacings from 5 mm to 50 mm, such as approximately 40 mm. In addition, IMD 910B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 910B may have a volume of three cubic centimeters (cm) or less, or 1.5 cubic cm or less, such as approximately 1.4 cubic cm.

In the example shown in FIG. 10B, once inserted subcutaneously within the patient, outer surface of cover 942 faces outward, toward the skin of the patient. In addition, as shown in FIG. 10B, proximal end 946 and distal end 948 are rounded to reduce discomfort and irritation to surrounding tissue once inserted under the skin of the patient. In addition, edges of IMD 910B may be rounded.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A system includes an implantable medical device comprising one or more sensors, and configured to receive one or more signals from the sensors, to determine a plurality of detected patient metrics based at least in part on the received signals; processing circuitry configured to generate higher resolution diagnostic information based on at least one of the plurality of patient metrics during an initiated period of time, wherein the initiated period of time is initiated based on one or more of an intervention being provided to the patient and receiving a user input, and the higher resolution diagnostic information comprises at least one of the plurality of patient metrics and is indicative of heart recovery; and communication circuitry configured to transmit the higher resolution diagnostic information to indicate a degree of recovery for one or more patient metrics indicative of heart recovery.

Example 2: The system of example 1, wherein the processing circuitry is further configured to generate the degree of recovery for one or more patient metrics based on the generated higher resolution diagnostic information.

Example 3: The system of any of examples 1 and 2, wherein the processing circuitry is further configured to generate a degree of heart recovery based on the generated higher resolution diagnostic information.

Example 4: The system of any of examples 1 through 3, wherein the at least one patient metric comprises at least one of fluid level, heart rate, respiration rate, activity, temperature, heart sounds, oxygenation, amplitude of S3 heart sounds, amplitude of S1 heart sounds, tissue oxygenation, and R-wave morphological characteristics.

Example 5: The system of any of examples 1 through 4, wherein the higher resolution diagnostic information is sampled at a sampling rate by the implantable medical device that is higher than a sampling rate of the implantable medical device before the initiated period of time is initiated.

Example 6: The system of any of examples 1 through 5, wherein the higher resolution diagnostic information is sampled with a quantity of sensors by the implantable medical device that is greater than a quantity of sensors of the implantable medical device before the initiated period of time is initiated.

Example 7: The system of any of examples 1 through 6, wherein the processing circuitry is further configured to: generate lower resolution diagnostic information based on at least one of the plurality of patient metrics before the initiated period of time; and switch from generating lower resolution diagnostic information to generating of higher resolution diagnostic information during the initiated period of time in response to receiving at least one of an indication the intervention is provided to the patient and a user input.

Example 8: The system of example 7, wherein the processing circuitry is further configured to switch from generating higher resolution diagnostic information during the initiated period of time to generating lower resolution diagnostic information in response to receiving an indication the initiated period of time ends.

Example 9: The system of any of examples 7 and 8, wherein the higher resolution diagnostic information is generated at least at a five times greater sampling rate than the lower resolution diagnostic information is generated.

Example 10: The system of any of examples 7 through 9, wherein the higher resolution diagnostic information is generated at least at a ten times greater sampling rate than the lower resolution diagnostic information is generated.

Example 11: The system of any of examples 7 through 10, wherein the higher resolution diagnostic information is generated from at least two times more sensors than the lower resolution diagnostic information is generated.

Example 12: The system of example 11, wherein the processing circuitry is further configured to: compare each of the plurality of detected patient metrics detected at an end of the initiated period of time to a respective detected patient metric detected at a beginning of the initiated period of time; and automatically generate a degree of heart recovery based on the comparison, wherein the degree of heart recovery is indicative of an amount the heart recovered over the initiated period of time.

Example 13: The system of any of examples 1 through 12, wherein the processing circuitry is further configured to: generate an average of each of the plurality of detected patient metrics detected over the initiated period of time; compare each of the averages to a respective detected patient metric detected at a beginning of the initiated period of time; and automatically generate a degree of heart recovery based on the comparison, wherein the degree of heart recovery is indicative of an amount the heart recovered over the initiated period of time.

Example 14: The system of any of examples 1 through 13, wherein the processing circuitry is further configured to: generate an average of each of the plurality of detected patient metrics detected over a second period of time, the second period of time beginning after the initiated period of time is initiated; compare each of the averages to a respective detected patient metric detected at a beginning of the period of time; and automatically generate a degree of heart recovery based on the comparison, wherein the degree of heart recovery is indicative of an amount the heart recovered over the period of time.

Example 15: The system of any of examples 1 through 14, wherein the processing circuitry is further configured to: generate a rate of change of each of the plurality of detected patient metrics detected over the initiated period of time; compare each of the rates of change to a respective threshold level; and automatically generating a degree of patient metric recovery of each of the plurality of automatically detected patient metrics based on the comparison, wherein the degree of patient metric recovery is indicative of an amount each parameter recovered over the initiated period of time.

Example 16: The system of example 15, wherein the processing circuitry is further configured to: automatically generate a degree of heart recovery based on the generated degrees of patient metric recovery, wherein the degree of heart recovery is indicative of an amount the heart recovered over the initiated period of time.

Example 17: The system of any of examples 1 through 16, wherein the processing circuitry is further configured to generate a treatment recommendation based on the degree of recovery.

Example 18: The system of example 17, wherein the treatment recommendation is further based on a type of intervention provided to the patient and a length of the initiated period of time.

Example 19: The system of any of examples 17 and 18, wherein the treatment recommendation includes one or more of medication recommendation, change in activity recommendation, and diet recommendation.

Example 20: A method includes obtaining a plurality of detected patient metrics by an implantable medical device of a patient; generating higher resolution diagnostic information of the patient during an initiated period of time, wherein the initiated period of time is initiated based on one or more of an intervention being provided to the patient and receiving a user input, and the higher resolution diagnostic information comprises at least one of the plurality of patient metrics and is indicative of heart recovery; and transmitting the generated higher resolution diagnostic information to indicate a degree of recovery for one or more patient metrics indicative of heart recovery.

Example 21: The method of example 20, further comprising generating a degree of recovery for one or more patient metrics based on the generated higher resolution diagnostic information.

Example 22: The method of any of examples 20 and 21, further comprising generating a degree of heart recovery based on the generated higher resolution diagnostic information.

Example 23: The method of any of examples 20 through 22, wherein the at least one patient metric comprises at least one of fluid level, heart rate, respiration rate, activity, temperature, heart sounds, oxygenation, amplitude of S3 heart sounds, amplitude of S1 heart sounds, tissue oxygenation, and R-wave morphological characteristics.

Example 24: The method of any of examples 20 through 23, wherein the higher resolution diagnostic information is sampled at a sampling rate by the implantable medical device that is higher than a sampling rate of the implantable medical device before the initiated period of time is initiated.

Example 25: The method of any of examples 20 through 24, wherein the higher resolution diagnostic information is sampled with a quantity of sensors by the implantable medical device that is greater than a quantity of sensors of the implantable medical device before the initiated period of time is initiated.

Example 26: The method of any of examples 20 through 25, further includes generating lower resolution diagnostic information based on at least one of the plurality of patient metrics before the initiated period of time; and switching from generating lower resolution diagnostic information to the generating of higher resolution diagnostic information during the initiated period of time in response to receiving at least one of an indication the intervention is provided to the patient and a user input.

Example 27: The method of example 26, further includes switching from the generating higher resolution diagnostic information during the initiated period of time to generating lower resolution diagnostic information in response to receiving an indication the initiated period of time ends.

Example 28: The method of any of examples 26 and 27, wherein the higher resolution diagnostic information is generated at least at a five times greater sampling rate than the lower resolution diagnostic information is generated.

Example 29: The method of any of examples 26 through 28, wherein the higher resolution diagnostic information is generated at least at a ten times greater sampling rate than the lower resolution diagnostic information is generated.

Example 30: The method of any of examples 26 through 29, wherein the higher resolution diagnostic information is generated from at least two times more sensors than the lower resolution diagnostic information is generated.

Example 31: The method of any of examples 20 through 30, further includes comparing each of the plurality of detected patient metrics detected at an end of the initiated period of time to a respective detected patient metric detected at a beginning of the initiated period of time; and automatically generating a degree of heart recovery based on the comparison, wherein the degree of heart recovery is indicative of an amount the heart recovered over the initiated period of time.

Example 32: The method of any of examples 20 through 31, further includes generating an average of each of the plurality of detected patient metrics detected over the initiated period of time; comparing each of the averages to a respective detected patient metric detected at a beginning of the initiated period of time; and automatically generating a degree of heart recovery based on the comparison, wherein the degree of heart recovery is indicative of an amount the heart recovered over the initiated period of time.

Example 33: The method of any of examples 20 through 32, further includes generating an average of each of the plurality of detected patient metrics detected over a second period of time, the second period of time beginning after the initiated period of time is initiated; comparing each of the averages to a respective detected patient metric detected at a beginning of the period of time; and automatically generating a degree of heart recovery based on the comparison, wherein the degree of heart recovery is indicative of an amount the heart recovered over the period of time.

Example 34: The method of any of examples 20 through 33, further includes generating a rate of change of each of the plurality of detected patient metrics detected over the initiated period of time; comparing each of the rates of change to a respective threshold level; and automatically generating a degree of patient metric recovery of each of the plurality of detected patient metrics based on the comparison, wherein the degree of patient metric recovery is indicative of an amount each parameter recovered over the initiated period of time.

Example 35: The method of example 34, further includes automatically generating a degree of heart recovery based on the generated degrees of patient metric recovery, wherein the degree of heart recovery is indicative of an amount the heart recovered over the initiated period of time.

Example 36: The method of any of examples 20 through 35, further comprising generating a degree of heart recovery with a probability model based on the plurality of generated patient metrics.

Example 37: The method of any of examples 1 through 36, further includes generating a treatment recommendation based on the degree of recovery.

Example 38: The method of example 37, wherein the treatment recommendation is further based on a type of intervention provided to the patient and a length of the initiated period of time.

Example 39: The method of any of examples 37 and 38, wherein the treatment recommendation includes one or more of medication recommendation, change in activity recommendation, and diet recommendation.

Example 40: A non-transitory computer-readable storage medium includes store a plurality of detected patient metrics within an implantable medical device of a patient; generate higher resolution diagnostic information of the patient during an initiated period of time, wherein the initiated period of time is initiated based on one or more of an intervention being provided to the patient and receiving a user input, and the higher resolution diagnostic information comprises at least one of the plurality of patient metrics and is indicative of heart recovery; and transmit the generated higher resolution diagnostic information to a display to indicate a degree of recovery for a respective patient metric indicative of heart recovery.

Various examples have been described. These and other examples are within the scope of the following claims.

What is claimed is:

1. A system comprising:
an implantable medical device comprising one or more sensors, and configured to receive one or more signals from the sensors, to determine a plurality of detected patient metrics based at least in part on the received signals;
processing circuitry configured to:
initiate a period of time based on an intervention being provided to the patient;
generate higher resolution diagnostic information based on at least one of the plurality of patient metrics during the initiated period of time, wherein the higher resolution diagnostic information comprises at least one of the plurality of patient metrics and is indicative of heart recovery, and wherein, to generate the higher resolution diagnostic information, the processing circuitry is configured to cause the implantable medical device to sample the at least one of the plurality of patient metrics at a sampling rate that is higher than a sampling rate of the at least one of the plurality of patient metrics before the initiated period of time;
generate an average of each of the plurality of detected patient metrics detected over the initiated period of time;
compare each of the averages to a respective detected patient metric detected at a beginning of the initiated period of time; and
automatically generate a degree of heart recovery based on the comparison, wherein the degree of heart recovery is indicative of an amount the heart recovered over the initiated period of time, and
communication circuitry configured to transmit the higher resolution diagnostic information and the degree of heart recovery.

2. The system of claim 1, wherein the at least one patient metric comprises at least one of fluid level, heart rate, respiration rate, activity, temperature, heart sounds, oxygenation, amplitude of S3 heart sounds, amplitude of S1 heart sounds, tissue oxygenation, and R-wave morphological characteristics.

3. The system of claim 1, wherein the higher resolution diagnostic information is sampled with a quantity of sensors by the implantable medical device that is greater than a quantity of sensors of the implantable medical device before the initiated period of time is initiated.

4. The system of claim 1, wherein the processing circuitry is further configured to:
generate lower resolution diagnostic information based on at least one of the plurality of patient metrics before the initiated period of time; and
switch from generating lower resolution diagnostic information to generating of higher resolution diagnostic information during the initiated period of time in response to receiving an indication the intervention is provided to the patient.

5. The system of claim 4, wherein the processing circuitry is further configured to switch from generating higher resolution diagnostic information during the initiated period of time to generating lower resolution diagnostic information in response to receiving an indication the initiated period of time ends.

6. The system of claim 4, wherein the higher resolution diagnostic information is generated at least at a five times greater sampling rate than the lower resolution diagnostic information is generated.

7. The system of claim 4, wherein the higher resolution diagnostic information is generated at least at a ten times greater sampling rate than the lower resolution diagnostic information is generated.

8. The system of claim 4, wherein the higher resolution diagnostic information is generated from at least two times more sensors than the lower resolution diagnostic information is generated.

9. The system of claim 8, wherein the processing circuitry is further configured to:
compare each of the plurality of detected patient metrics detected at an end of the initiated period of time to a respective detected patient metric detected at a beginning of the initiated period of time; and
automatically generate the degree of heart recovery based on the comparison.

10. The system of claim 1, wherein the processing circuitry is further configured to:
generate an average of each of the plurality of detected patient metrics detected over a second period of time, the second period of time beginning after the initiated period of time is initiated;
compare each of the averages to a respective detected patient metric detected at a beginning of the initiated period of time; and
automatically generate the degree of heart recovery based on the comparison.

11. The system of claim 1, wherein the processing circuitry is further configured to:
generate a rate of change of each of the plurality of detected patient metrics detected over the initiated period of time;
compare each of the rates of change to a respective threshold level; and
automatically generating a degree of patient metric recovery of each of the plurality of automatically detected patient metrics based on the comparison, wherein the degree of patient metric recovery is indicative of an amount each parameter recovered over the initiated period of time.

12. The system of claim 11, wherein the processing circuitry is further configured to:
automatically generate the degree of heart recovery based on the generated degrees of patient metric recovery.

13. The system of claim 1, wherein the processing circuitry is further configured to generate a treatment recommendation based on the degree of heart recovery.

14. The system of claim 13, wherein the treatment recommendation is further based on a type of intervention provided to the patient and a length of the initiated period of time.

15. The system of claim 13, wherein the treatment recommendation includes one or more of medication recommendation, change in activity recommendation, and diet recommendation.

16. The system of claim 1, wherein the at least one patient metric comprises at least one of daily activity, night heart rate, or heart rate variability.

17. A method comprising:
obtaining a plurality of detected patient metrics by an implantable medical device of a patient;
initiating a period of time based on an intervention being provided to the patient;
causing the implantable medical device to sample at least one of the plurality of patient metrics at a sampling rate that is higher than a sampling rate of the at least one of the plurality of patient metrics before the initiated period of time to generate higher resolution diagnostic information of the patient during the initiated period of time, wherein the higher resolution diagnostic information comprises the at least one of the plurality of patient metrics and is indicative of heart recovery;
generating an average of each of the plurality of detected patient metrics detected over the initiated period of time;
comparing each of the averages to a respective detected patient metric detected at a beginning of the initiated period of time;
automatically generating a degree of heart recovery based on the comparison, wherein the degree of heart recovery is indicative of an amount the heart recovered over the initiated period of time, and
transmitting the generated higher resolution diagnostic information to indicate a degree of recovery for one or more patient metrics indicative of heart recovery.

18. The method of claim 17, wherein the at least one patient metric comprises at least one of fluid level, heart rate, respiration rate, activity, temperature, heart sounds, oxygenation, amplitude of S3 heart sounds, amplitude of S1 heart sounds, tissue oxygenation, and R-wave morphological characteristics.

19. The method of claim 17, further comprising:
generating lower resolution diagnostic information based on at least one of the plurality of patient metrics before the initiated period of time; and
switching from generating lower resolution diagnostic information to the generating of higher resolution diagnostic information during the initiated period of time in response to receiving an indication the intervention is provided to the patient.

20. The method of claim 17, further comprising:
comparing each of the plurality of detected patient metrics detected at an end of the initiated period of time to a respective detected patient metric detected at a beginning of the initiated period of time; and
automatically generating the degree of heart recovery based on the comparison.
